(19) United States
(12) Patent Application Publication
(10) Pub. No.: US 2025/0375556 A1
(43) Pub. Date: Dec. 11, 2025

(54) MEDICAL DEVICE AND METHOD FOR MANUFACTURING THE SAME

(71) Applicant: TERUMO KABUSHIKI KAISHA, Tokyo (JP)

(72) Inventor: Takeshi MATSUHASHI, Fujinomiya (JP)

(73) Assignee: TERUMO KABUSHIKI KAISHA, Tokyo (JP)

(21) Appl. No.: 19/307,609

(22) Filed: Aug. 22, 2025

Related U.S. Application Data
(63) Continuation of application No. PCT/JP2024/009006, filed on Mar. 8, 2024.

(30) Foreign Application Priority Data
Mar. 10, 2023 (JP) 2023-037955

(51) Int. Cl. A61L 31/10; A61L 31/14

(52) U.S. Cl. CPC A61L 31/10; A61L 31/14

(57) ABSTRACT

A medical device with improved lubrication retaining property includes a base layer, and a lubricating layer having a first layer formed on at least a part of the base layer, and a second layer formed on at least a part of the first layer, in which the first layer includes a first copolymer having a structural unit derived from a hydrophilic monomer and a structural unit having an epoxy group, and has a swelling ratio of more than 70% and less than 1000%, and the second layer includes a second copolymer having a structural unit having an alkyl vinyl ether group and a structural unit having a carboxyl group or a salt or ester thereof.

### CROSS-REFERENCES TO RELATED APPLICATIONS

This application is a continuation of International Patent Application No. PCT/JP2024/009006 filed on Mar. 8, 2024, which claims priority to Japanese Patent Application No. 2023-037955 filed on Mar. 10, 2023, the entire content of both of which is incorporated herein by reference.

### TECHNOLOGICAL FIELD

The present disclosure generally relates to a medical device and a method for manufacturing the same.

### BACKGROUND DISCUSSION

Medical devices inserted into a living body, such as a catheter and a guide wire, are required to exhibit excellent lubricating properties in order to reduce damage to tissues such as a blood vessel and to improve the operability of an operating surgeon. For this reason, a method for covering a base layer surface of a medical device with a hydrophilic polymer having a lubricating property has been developed and put to practical use. On the other hand, it is also important that such a medical device can retain the lubricating property on the surface of the base layer at the time of use by an operating surgeon in order to maintain operability of the operating surgeon. Therefore, coating with a hydrophilic polymer is required not only to have excellent lubricating property but also to have durability against loads such as wear and abrasion.

From such a viewpoint, JP S60-259269 A discloses a medical device in which a maleic anhydride-based polymer substance is covalently bonded to the surface of a base material, constituting a medical device via a reactive functional group to form a surface lubricating layer on the surface of the base layer.

### SUMMARY

According to the technique disclosed in JP S60-259269 A, a surface lubricating layer exhibiting a good lubricating property can be immobilized to a base material. On the other hand, in recent years, an approach of treating a lesion site through a complicated living body lumen has been spreading with the reduction in size and diameter of medical devices. In addition, as the medical procedure becomes complicated, the operation of the medical device may take a longer time. Therefore, in order to maintain good operability of the medical device for a longer time, even in the case of treating a lesion site through a complicated living body lumen, there is a demand for a technique for further improving a lubrication retaining property (sliding durability) of the surface of the medical device as compared with the prior art. More specifically, there is a demand for a medical device having excellent sliding durability and capable of retaining a lubricating property which is high even when sliding of the surface of the medical device is repeated.

Disclosed here is a technique capable of improving the lubrication retaining property (sliding durability).

The inventors have intensively studied to solve the problems. As a result, the inventors have found that the above problems can be solved by sequentially disposing a layer including a copolymer that exhibits a specific swelling ratio and has a specific structural unit and a layer including a copolymer having a specific structural unit on a base material. The medical device disclosed here was completed based on the above findings.

According to one aspect, (1) a medical device including: a base layer; and a lubricating layer having a first layer formed on at least a part of the base layer and a second layer formed on at least a part of the first layer, in which the first layer includes a first copolymer having a structural unit derived from a hydrophilic monomer and a structural unit having an epoxy group, and has a swelling ratio of more than 70% and less than 1000%, and the second layer includes a second copolymer having a structural unit having an alkyl vinyl ether group and a structural unit having a carboxyl group or a salt or ester thereof.

According to at least some embodiments, (2) in the medical device of the above (1), it is preferable that
a composition of the structural unit derived from a hydrophilic monomer and the structural unit having an epoxy group in the first copolymer (a molar ratio of the structural unit derived from a hydrophilic monomer: the structural unit having an epoxy group) is 3:1 to 70:1.

In accordance with at least some embodiments, (3) in the medical device of the above (1) or (2), it is preferable
that the first copolymer has a structural unit derived from a hydrophilic monomer having an amino group (—N(R1)(R2); R1 and R2 each independently represent a hydrogen atom or an unsubstituted or substituted alkyl group having 1 to 4 carbon atoms) or an alkylene glycol chain and a structural unit having an epoxy group.

(4) In the medical device according to any one of the above (1) to (3), it is preferable that the structural unit derived from a hydrophilic monomer is a structural unit derived from at least one monomer selected from the group consisting of acrylamide, N-methyl acrylamide, N,N-dimethylacrylamide (DMAA), N-ethylacrylamide, N,N-diethylacrylamide (DEAA), N,N-dimethylaminopropylacrylamide, N,N-dimethylaminopropylmethacrylamide, 2-acrylamide-2-methylpropanesulfonic acid, N-(2-hydroxyethyl) acrylamide, N-(2-hydroxypropyl) acrylamide, N-(2-hydroxybutyl) acrylamide, polyethylene glycol monoacrylate, polyethylene glycol monomethacrylate, alkoxypolyethylene glycol monoacrylate, and alkoxypolyethylene glycol monomethacrylate.

(5) In the medical device according to any one of the above (1) to (4), it is preferable that the structural unit having an epoxy group is a structural unit derived from at least one monomer selected from the group consisting of glycidyl acrylate, glycidyl methacrylate (GMA), 3,4-epoxycyclohexyl methyl acrylate, 3,4-epoxycyclohexyl methyl methacrylate, β-methylglycidyl acrylate, and β-methylglycidyl methacrylate.

(6) In the medical device according to any one of the above (1) to (5), it is preferable that the structural unit having a carboxyl group or a salt or ester thereof includes at least one of a structural unit represented by the following formula (1) and a structural unit represented by the following formula (2):

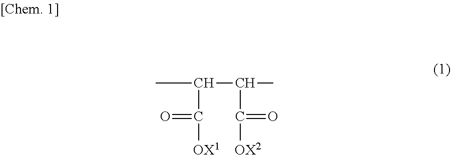

in the above formula (1), X1 represents a hydrogen atom, an alkali metal or an alkaline earth metal, and X2 represents a hydrogen atom, an alkali metal, an alkaline earth metal, or a straight chain alkyl group having 1 to 24 carbon atoms or branched chain alkyl group having 3 to 24 carbon atoms,

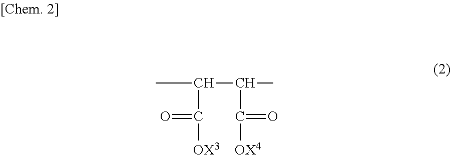

in the above formula (2), X3 represents a hydrogen atom, an alkali metal or an alkaline earth metal, and X4 represents a hydrogen atom, an alkali metal, or an alkaline earth metal.

(7) In the medical device according to any one of the above (1) to (6), it is preferable that the structural unit having an alkyl vinyl ether group is represented by the following formula (3):

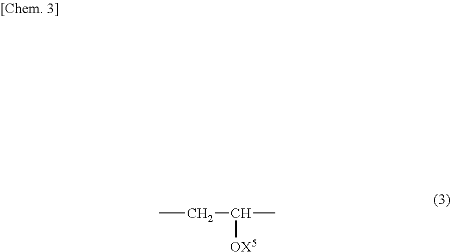

in the above formula (3), X5 represents a straight chain alkyl group having 1 to 24 carbon atoms or branched chain alkyl group having 3 to 24 carbon atoms.

(8) In the medical device according to any one of the above (1) to (7), it is preferable that the lubricating layer has a region in which the first copolymer and the second copolymer are bonded to each other.

(9) In the medical device according to any one of the above (1) to (8), it is preferable that the medical device is a catheter, a stent, or a guide wire.

According to another aspect, (10) a method for manufacturing a medical device, the method including: applying a first coating liquid including a first copolymer having a structural unit derived from a hydrophilic monomer and a structural unit having an epoxy group and a solvent to at least a part of a base layer to form a first precursor layer on at least a part of the base layer; applying a second coating liquid including a second copolymer having a structural unit having an alkyl vinyl ether group and a structural unit having a carboxyl group or a salt or ester thereof and a solvent to at least a part of the first precursor layer to obtain an intermediate laminated body, in which a second precursor layer is formed on at least a part of the first precursor layer; and irradiating the intermediate laminated body with an electron beam.

Here, in the embodiments of the present disclosure, (11) in the manufacture method above (10), it is preferable that the intermediate laminated body is irradiated with an electron beam at an irradiation dose of 30 to 500 kGy.

According to another aspect, a medical device comprises: a base layer; and two additional layers that together constitute a lubricating layer and that are successively applied to the base layer so that the two additional layers overlie the base layer. The two additional layers that are successively applied to the base layer comprise a first layer and a second layer, with at least a part of the first layer being in contact with the base layer and being between the base layer and the second layer, and at least a part of the second layer being in contact with the first layer. The first layer comprises a first copolymer, with the first copolymer comprising a structural unit derived from a hydrophilic monomer and a structural unit having an epoxy group, and the first layer having a swelling ratio of at least 190% and less than 1000%. The second layer comprises a second copolymer, with the second copolymer comprising a structural unit having an alkyl vinyl ether group and a structural unit having a carboxyl group or a salt or ester thereof.

### BRIEF DESCRIPTION OF THE DRAWINGS

FIG. 1 is a schematic diagram of a test device (friction tester) for evaluating a lubricating property/lubrication retaining property. In FIG. 1, the reference numeral 1 indicates water; the reference numeral 2 indicates a petri dish; the reference numeral 3 indicates a sample (medical device); the reference numeral 4 indicates a terminal; the reference numeral 5 indicates a load; the reference numeral 6 indicates a moving table; and the reference numeral 10 indicates a friction tester.

FIG. 2 is a partial cross-sectional view schematically illustrating a lamination structure of a surface of a representative embodiment of a medical device manufactured by the method according to the present disclosure. In FIG. 2, reference numeral 100 indicates a medical device; reference numeral 101 indicates a base layer; reference numeral 102 indicates a lubricating layer; reference numeral 102a indicates the first layer; and reference numeral 102b indicates the second layer, respectively.

FIG. 3 is a partial cross-sectional view schematically illustrating a different structure example of the lamination structure of the surface, as an application example of the embodiment of FIG. 2. In FIG. 3, reference numeral 100 indicates a medical device; reference numeral 101 indicates a base layer; reference numeral 101a indicates a base layer core portion; reference numeral 101b indicates a base material surface layer; reference numeral 102 indicates a lubricating layer; reference numeral 102a indicates a first layer; and reference numeral 102b indicates a second layer, respectively.

### DETAILED DESCRIPTION

A first aspect of the disclosure here provides a medical device including: a base layer; and a lubricating layer having a first layer formed on at least a part of the base layer and a second layer formed on at least a part of the first layer, in which the first layer includes a first copolymer having a structural unit derived from a hydrophilic monomer and a structural unit having an epoxy group, and has a swelling ratio of more than 70% and less than 1000%, and the second layer includes a second copolymer having a structural unit having an alkyl vinyl ether group and a structural unit having a carboxyl group or a salt or ester thereof.

Another aspect of the disclosure involves a method for manufacturing a medical device that includes: applying a first coating liquid including a first copolymer having a structural unit derived from a hydrophilic monomer and a structural unit having an epoxy group and a solvent to at least a part of a base layer to form a first precursor layer on at least a part of the base layer; applying a second coating liquid including a second copolymer having a structural unit having an alkyl vinyl ether group and a structural unit having a carboxyl group or a salt or ester thereof and a solvent to at least a part of the first precursor layer to obtain an intermediate laminated body, in which a second precursor layer is formed on at least a part of the first precursor layer; and irradiating the intermediate laminated body with an electron beam.

According to the present disclosure, a medical device including a lubricating layer having an excellent lubrication retaining property (sliding durability) is provided.

According to the present disclosure, a technique capable of improving the lubrication retaining property (sliding durability) is provided. Therefore, the medical device having the above configuration has a lubricating layer capable of exhibiting the excellent lubrication retaining property (sliding durability). In addition, the medical device having the above configuration has a lubricating layer having the excellent lubricating property.

In the present description, the lubrication retaining property (sliding durability) is also simply referred to as “durability” or a “lubrication retaining property”.

Hereinafter, preferred embodiments of the present disclosure will be described. The present disclosure is not limited only to the following embodiments, and various modifications can be made within the scope of claims. In addition, the embodiments described in the present description can be arbitrarily combined with each other to form another embodiment. Each drawing is exaggerated for convenience of description, and dimensional ratios of each component in each drawing may be different from actual ones. In addition, in a case where embodiments are described with reference to the drawing, similar elements are indicated by the same reference numeral in the description of the drawing, and a detailed description of such elements is not repeated.

Throughout the present description, expression of a singular should be understood to include also a concept of a plural thereof unless otherwise stated. Thus, an article of a singular (for example, “a”, “an”, and “the” in English) should be understood to include also a concept of a plural thereof unless otherwise stated. In addition, terms used in the present description should be understood to be used in a sense commonly used in the art unless otherwise stated. Therefore, unless otherwise defined, all technical and scientific terms used in the present description have the same meanings as commonly understood by a person skilled in the art to which the present disclosure belongs. In a case of contradiction, priority is given to the present description (including definitions).

In the present description, when a structural unit is defined to be “derived” from a certain monomer, it means that the structural unit is a structural unit generated by cleavage of one bond of a polymerizable unsaturated double bond of the corresponding monomer.

In the present description, the term “(meth)acryl” represents both acryl and methacryl. Therefore, for example, the term “(meth)acrylic acid” encompasses both acrylic acid and methacrylic acid. Similarly, the term “(meth)acryloyl” refers to both acryloyl and methacryloyl. Therefore, for example, the term “(meth)acryloyl group” refers to both an acryloyl group and a methacryloyl group.

In the present description, a range from “X to Y” includes X and Y and indicates “X or more and Y or less”. In the present description, “A and/or B” means at least one of A and B and includes both A and B or either A or B. Unless otherwise specified, operations and measurements of physical properties and the like are performed at room temperature (20 to 25° C.) and at relative humidity of 40 to 60% RH.

### <Medical Device>

A medical device includes a base layer and a lubricating layer formed on at least a part of the base layer. The lubricating layer includes a first layer and a second layer formed on at least a part of the first layer. The first layer includes a first copolymer having a structural unit derived from a hydrophilic monomer and a structural unit having an epoxy group and has a swelling ratio of more than 70% and less than 1000%. Here, the first copolymer has an epoxy group. When a coating liquid (first coating liquid) for forming the first layer and a coating liquid (second coating liquid) for forming the second layer are sequentially applied to the base layer and then subjected to an active energy ray irradiation treatment, such as electron beam irradiation, the epoxy group is cleaved, and the first copolymer and the second copolymer are bonded (crosslinked), the first copolymer and a material constituting the base layer are bonded (crosslinked), and the first copolymers are bonded (crosslinked) to each other. Therefore, the base layer and the second layer can be firmly bonded (immobilized) via the first layer, and the film strength of the first layer is increased. The first copolymer has a structural unit derived from a hydrophilic monomer. Thus, the first layer has a swelling property when brought into contact with an aqueous medium (in particular water alone or a combination of water and a lower alcohol) and exhibits a specific swelling ratio. A lower alcohol is an alcohol with a small number of carbon atoms in its hydrocarbon chain. When the second layer is formed using a coating liquid (second coating liquid) including the second copolymer and an aqueous medium, the first precursor layer swells when the second coating liquid is applied to the first precursor layer (the layer formed by the first coating liquid), and the second copolymer easily penetrates into the swelled first precursor layer. According to this configuration, the first copolymer and the second copolymer come into contact with each other at more sites (therefore, the number of reaction points between the first copolymer and the second copolymer is larger, and the reaction points are closer to one another). For this reason, the first copolymer and the second copolymer can react at more reaction points by an active energy ray irradiation treatment, such as electron beam irradiation, and more regions in which the first copolymer and the second copolymer are bonded (crosslinked) to each other can be formed. Therefore, the first layer can be more firmly bonded (immobilized) to the second layer. Therefore, the medical device having the lubricating layer according to the disclosure exhibits excellent durability. In addition, the medical device having a lubricating layer according to the present disclosure has an excellent lubricating property.

The above mechanism is a presumption and does not limit the technical scope of the present disclosure.

Hereinafter, a preferred embodiment of a medical device according to the present disclosure will be described with reference to the accompanying drawings.

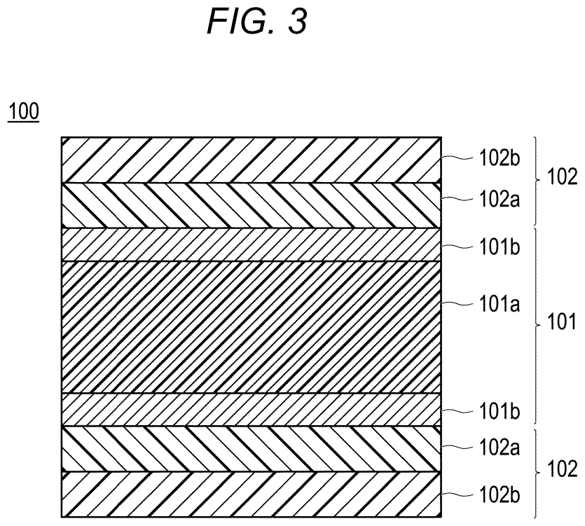

FIG. 2 is a partial cross-sectional view schematically showing a lamination structure of a surface of a representative embodiment of the medical device according to the present disclosure (hereinafter, it can also be simply referred to as a “medical device”). FIG. 3 is a partial cross-sectional view schematically illustrating a different configuration example of the lamination structure of the surface as an application example of the present embodiment.

As illustrated in FIG. 2 and FIG. 3, the medical device 100 of the present embodiment includes a base layer 101 and a lubricating layer 102. The lubricating layer 102 has a first layer 102a formed on at least a part of the base layer 101 (the drawings illustrate an example in which the layer is immobilized to the entire surface (whole surface) of the base layer 101) and a second layer 102b formed on at least a part of the first layer 102a (the drawings illustrate an example in which the layer is immobilized to the entire surface (whole surface) of the first layer 102a).

Hereinafter, each configuration or portion of the medical device of the present embodiment will be described.

### [Base Layer (Base Material)]

The base layer may be composed of any suitable material and can be appropriately selected according to the application. Specifically, examples of the material composing (forming) the base layer 101 include a metal material, a polymer material, and ceramics. Here, the base layer 101 may have a structure in which the entire base layer 101 (all) is composed (formed) of any of the above materials as shown in FIG. 2. Alternatively, as shown in FIG. 3, the surface of the base layer core portion 101a composed (formed) of any of the above materials can be covered (coated) with any other of the above materials by an appropriate method to constitute (form) the base material surface layer 101b. Examples of the latter case include a base layer in which the base material surface layer 101b is formed by covering (coating) a surface of the base layer core portion 101a formed from a resin material and the like, with a metal material through an appropriate method (for example, a method in the related art such as plating, metal deposition, and sputtering). Examples of the latter case further include a base layer in which the base material surface layer 101b is formed by covering (coating) a surface of the base layer core portion 101a formed from a hard reinforcing material, such as a metal material and a ceramic material, with a polymer material softer than the reinforcing material, such as a metal material, through an appropriate method (for example, a method in the related art such as immersing (dipping), spraying, and coating and printing). Examples of the latter case further include a base layer formed by performing complexation (appropriate reaction processing) of a reinforcing material for the base layer core portion 101a and a polymer material for the base material surface layer 101b. Accordingly, the base layer core portion 101a may be a multilayer structure obtained by laminating different materials in multiple layers, or a structure (for example, a complex) obtained by connecting members formed from different materials for each part of the medical device, or the like. In addition, another middle or intermediate layer (not shown) may be further formed between the base layer core portion 101a and the base material surface layer 101b. Furthermore, the base material surface layer 101b may also be a multilayer structure obtained by laminating different materials in multiple layers, or a structure (for example, a complex) obtained by connecting members formed from different materials for each part of the medical device, or the like.

Among the materials constituting (forming) the base layer 101, the type of metal material is not particularly limited, and any metal materials generally used for medical devices such as a catheter, a stent, and a guide wire can be used. Specific examples thereof include various stainless steel (SUS) such as SUS304, SUS316, SUS316L, SUS420J2, and SUS630, and various alloys such as gold, platinum, silver, copper, nickel, cobalt, titanium, iron, aluminum, tin, or a nickel-titanium (Ni—Ti) alloy, a nickel-cobalt (Ni—Co) alloy, a cobalt-chromium (Co—Cr) alloy, and a zinc-tungsten (Zn—W) alloy. These may be used singly or in combination of two or more kinds thereof. As the above metal material, a suitable metal material may be appropriately selected for the base layer intended to be used for a catheter, a stent, and a guide wire.

In addition, among the materials constituting (forming) the base layer 101, the type of polymer material is not particularly limited, and any polymer materials generally used for medical devices such as a catheter, a stent, and a guide wire are used. Specific examples thereof include polyamide resins such as nylon (including a nylon elastomer) and polyamide-based elastomers, polyethylene such as linear low-density polyethylene (LLDPE), low-density polyethylene (LDPE), high-density polyethylene (HDPE) and modified polyethylene, polyolefin resins such as polypropylene, polyester resins such as polyethylene terephthalate (PET), styrene resins such as polystyrene, cyclic polyolefin resins, modified polyolefin resins, epoxy resins, urethane resins (including a polyurethane elastomer), diallyl phthalate resins (allyl resins), polycarbonate resins, fluororesins, amino resins (urea resins, melamine resins, benzoguanamine resins), acrylic resins, polyacetal resins, vinyl acetate resins, phenol resins, vinyl chloride resins (polyvinyl chloride (PVC)), polytetrafluoroethylene (PTFE), silicone resins and polyether resins such as polyether ether ketone (PEEK), and polyimide resins. These polymer materials may be used singly or in combination of two or more kinds thereof. As the above polymer material, a suitable polymer material may be appropriately selected for the base layer intended to be used for a catheter, a stent, and a guide wire.

Preferably, the base layer 101 includes or is composed of a polymer material, or the base material surface layer includes or is composed of a polymer material. More preferably, the base layer 101 is composed of a polymer material, or the base material surface layer is composed of a polymer material.

The shape of the above base layer is not particularly limited and is appropriately selected according to a use mode such as a sheet shape, a linear shape (wire), or a tubular shape.

### [Lubricating Layer]

As shown in FIG. 2, the lubricating layer 102 is formed (supported) on at least a part of the base layer (base material) 101. Here, the reason why the lubricating layer 102 is formed (supported) on at least a part of the surface of the base layer 101 is that, in medical devices such as a catheter, a stent, and a guide wire, and an indwelling needle to be used, it is not always necessary that whole surface (the entire surface) of these medical devices has the lubricating property in a wet state, and it may be sufficient that the lubricating layer is supported only on a surface portion (which may be a part or all) where the surface is required to have the lubricating property in a wet state. Thus, as described above, the lubricating layer includes: a form formed so as to cover only the entire one surface of the base layer 101 as shown in FIG. 2; a form formed so as to cover the entire both surfaces of the base layer 101 as shown in FIG. 3; a form formed so as to cover a part of both surfaces of the substrate layer in the same or different form; and a form formed so as to cover a part of one surface of the base layer 101.

In FIGS. 2 and 3, the lubricating layer 102 has a first layer 102a and a second layer 102b.

### (First Layer)

The first layer 102a is formed on at least a part of the base layer 101. Here, the first layer 102a may be formed on a surface (which may be a part or all) of the base layer 101, the surface of which is required to have the lubricating property in a wet state, and the necessity of forming the first layer 102a is appropriately selected according to an application of a medical device such as a catheter, a guide wire, or an indwelling needle to be used.

The first layer 102a includes a first copolymer having the structural unit derived from a hydrophilic monomer and the structural unit having an epoxy group. Due to the presence of the epoxy group, the base layer 101 and the second layer 102b can be firmly bonded via the first layer 102a. In addition, the film strength of the first layer 102a is increased. Due to the presence of the structural unit derived from a hydrophilic monomer, the first layer 102a can exhibit a specific swelling ratio.

The first layer 102a may have a one-layer form or a laminated form of two or more layers. Preferably, the first layer 102a is in one-layer form.

The first layer 102a has a swelling ratio of more than 70% and less than 1000%. Here, if the swelling ratio of the first layer 102a is 70% or less, the swelling of the first layer (first precursor layer) is insufficient, and a sufficient amount of the second copolymer cannot penetrate into the first precursor layer at the time of swelling. Therefore, the first copolymer and the second copolymer cannot be sufficiently bonded (crosslinked), and the lubricating layer 102 is poor in durability. In addition, if the swelling ratio of the first layer is 1000% or more, the first layer 102a dissolves or the first layer swells too much to maintain sufficient strength, and the lubricating layer 102 also has poor durability. In consideration of the effect (particularly durability) and the like, the swelling ratio of the first layer 102a is preferably 100% or more and less than 950%, more preferably 190% or more and 930% or less, still more preferably 220% or more and less than 930%, even more preferably 320% or more and 920% or less, particularly preferably 500% or more and 900% or less, and most preferably 670% or more and 900% or less. In the present description, as the “swelling ratio (%)”, a value measured according to the following method is adopted.

### [Measurement of Swelling Ratio]

The swelling ratio of the first layer was measured by the following method.

### (Production of Sample for Measuring Swelling Ratio)

The (co)polymer used for formation of the first layer was dissolved in acetone so as to have a concentration of 6% by mass to prepare a copolymer solution. Next, 15 g of this copolymer solution was developed on a petri dish made of polytetrafluoroethylene (PTFE) material of 75 mm Φ. Furthermore, the petri dish was naturally dried for 3 hours and then dried under reduced pressure at room temperature (25° C.) for 24 hours to obtain a film sample. When two or more (co)polymers were used for formation of the first layer, a copolymer solution was prepared so that the total concentration of the (co)polymers was 6% by mass.

The film sample obtained as described above was cut into 3 cm×3 cm to obtain a measurement sample.

### (Measurement of Swelling Ratio)

The mass (E [g]) of the measurement sample produced above was measured, and then the sample was immersed in 100 mL of RO water weighed in a beaker at room temperature (25° C.) for 3 minutes to swell the measurement sample. Thereafter, the swollen measurement sample was taken out, and the surface water was wiped off using a paper towel, and then the mass (F [g]) thereof was measured. Based on the mass (E [g] and F [g]) of the measurement sample before and after swelling, the swelling ratio (%) was calculated according to the following formula. When the swelling ratio was 1000% or more or when the measurement sample was dissolved at the time of measuring the swelling ratio and measurement was not possible, it was determined as “dissolved”.

$$Swelling ratio [ \% ] = ( F - E ) \times 100 / E \qquad [ Math . 1 ]$$

The hydrophilic monomer constituting the first copolymer preferably has an ethylenically unsaturated group such as an acryloyl group (H₂C═CH—(C═O)—), a methacryloyl group (H₂C═C(CH₃)—(C═O)—), a vinyl group (H₂C═CH—), an isopropenyl group (H₂C═C(CH₃)—), or an allyl group (H₂C═CHCH₂—).

The hydrophilic monomer constituting the first copolymer preferably has a primary, secondary, or tertiary amino group or an alkylene glycol chain, more preferably has a secondary or tertiary amino group or an alkylene glycol chain, and particularly preferably has a tertiary amino group, from the viewpoint of appropriately controlling the swelling property. At this time, the amino group may have a substituent.

Here, in the formula: —N(R1)(R2) representing an amino group when the hydrophilic monomer has an amino group, R1 and R2 represent a hydrogen atom or an unsubstituted or substituted alkyl group having 1 to 5 carbon atoms. In the above formula, R1 and R2 may be the same or different. Examples of the alkyl group having 1 to 5 carbon atoms (unsubstituted form) include a methyl group, an ethyl group, a n-propyl group, an isopropyl group, a n-butyl group, an isobutyl group, a sec-butyl group, a tert-butyl group, a n-pentyl group, an isopentyl group, a tert-pentyl group, a neopentyl group, and a 1,2-dimethylpropyl group. R1 is preferably a hydrogen atom or a straight chain or branched chain alkyl group having 1 to 4 carbon atoms, more preferably a hydrogen atom, a straight chain alkyl group having 1 to 3 carbon atoms, or a branched chain alkyl group having 3 carbon atoms, still more preferably a hydrogen atom, a methyl group, or an ethyl group, particularly preferably a hydrogen atom or a methyl group, and most preferably a methyl group. R2 is preferably a hydrogen atom, a straight chain alkyl group having 1 to 4 carbon atoms, or a branched chain alkyl group having 3 to 4 carbon atoms, more preferably a hydrogen atom, a straight chain alkyl group having 1 to 3 carbon atoms, or a branched chain alkyl group having 3 carbon atoms, still more preferably a hydrogen atom, a methyl group, or an ethyl group, particularly preferably a hydrogen atom or a methyl group, and most preferably a methyl group. When the alkyl group has a substituent (R1 or R2 represents a substituted alkyl group having 1 to 5 carbon atoms), examples of the substituent include a hydroxyl group (—OH) and a sulfonic acid group (—SO₃H).

In a preferred embodiment of the present disclosure, the first copolymer has a structural unit derived from a hydrophilic monomer having an amino group (—N(R1)(R2); R1 and R2 each independently represent a hydrogen atom or an unsubstituted or substituted alkyl group having 1 to 5 carbon atoms) and/or an alkylene glycol chain and a structural unit having an epoxy group.

In a more preferred embodiment of the present disclosure, the first copolymer has a structural unit derived from a hydrophilic monomer having an amino group (—N(R1)(R2); R1 and R2 each independently represent a hydrogen atom or an unsubstituted or substituted alkyl group having 1 to 4 carbon atoms) or an alkylene glycol chain and a structural unit having an epoxy group. In a further preferred embodiment, the first copolymer has a structural unit derived from a hydrophilic monomer having an amino group (—N(R1)(R2); R1 and R2 each independently represent a hydrogen atom or an unsubstituted or substituted alkyl group having 1 to 3 carbon atoms) and a structural unit having an epoxy group. In a particularly preferred embodiment, the first copolymer has a structural unit derived from a hydrophilic monomer having an amino group (—N(R1)(R2); R1 and R2 each represent a hydrogen atom or a methyl group (particularly a methyl group)) and a structural unit having an epoxy group.

Examples of the hydrophilic monomer having an amino group include acrylamide, methacrylamide, N-methylacrylamide, N-methylmethacrylamide, N,N-dimethylacrylamide (DMAA), N,N-dimethylmethacrylamide, N-ethylacrylamide, N-ethylmethacrylamide, N,N-diethylacrylamide (DEAA), N,N-diethylmethacrylamide, N,N-dimethylaminopropylacrylamide, N,N-dimethylaminopropylmethacrylamide, N,N-dimethylaminoethylacrylate, N,N-dimethylaminoethylmethacrylate, 2-acrylamide-2-methylpropanesulfonic acid, N-(2-hydroxyethyl) acrylamide, N-(2-hydroxypropyl) acrylamide, and N-(2-hydroxybutyl) acrylamide. Among these hydrophilic monomers, from the viewpoint of imparting a specific swelling property, easiness of synthesis, operability, and the like, the hydrophilic monomer preferably includes at least one selected from the group consisting of acrylamide, N-methylacrylamide, N,N-dimethylacrylamide (DMAA), N-ethylacrylamide, N,N-diethylacrylamide (DEAA), N,N-dimethylaminopropylacrylamide, N,N-dimethylaminopropylmethacrylamide, 2-acrylamide-2-methylpropanesulfonic acid, N-(2-hydroxyethyl) acrylamide, N-(2-hydroxypropyl) acrylamide, and N-(2-hydroxybutyl) acrylamide, more preferably includes at least one selected from the group consisting of acrylamide, N,N-dimethylacrylamide, and N,N-diethylacrylamide, still more preferably acrylamide or N,N-dimethylacrylamide, and particularly preferably N,N-dimethylacrylamide.

When the hydrophilic monomer has an alkylene glycol chain, the hydrophilic monomer may have an ethylenically unsaturated group such as an acryloyl group (CH₂═CH—C(═O)—), a methacryloyl group (CH₂═C(CH₃)—C(═O)—), or a vinyl group (CH₂═CH—); and the structure represented by formula: -(repeating unit)ₙ-R. Here, the repeating unit has an alkylene oxide such as ethylene oxide (—OCH₂CH₂—), propylene oxide (—OCH₂CH₂CH₂—), propylene oxide (—OCH(CH₃)CH₂—, —OCH₂CH(CH₃)—) or the like. Among these, the repeating unit is preferably ethylene oxide, propylene oxide, or isopropylene oxide, more preferably ethylene oxide or propylene oxide, and particularly preferably ethylene oxide. The number of repeating units of the repeating unit (“n” in the formula: -(repeating unit)ₙ-R) is 2 or more, preferably 5 or more, and more preferably 7 or more. The number of repeating units of the repeating unit (n in the formula: -(repeating unit)ₙ-R) is 100 or less, preferably 90 or less, and more preferably 80 or less. Within such a range, the first layer 102a exhibits an appropriate swelling property. R in the above formula is not particularly limited. Examples thereof include a hydrogen atom, a straight chain alkoxy group having 1 to 30 carbon atoms, a branched chain alkoxy group having 3 to 30 carbon atoms, and a phenoxy group.

Here, the straight chain alkoxy group having 1 to 30 carbon atoms or the branched chain alkoxy group having 3 to 30 carbon atoms is not particularly limited, and examples thereof include a methoxy group, an ethoxy group, a propoxy group, an isopropoxy group, a butoxy group, a pentyloxy group, a hexyloxy group, a 2-ethylhexyloxy group, an octyloxy group, a nonyloxy group, a decyloxy group, an undecyloxy group, a dodecyloxy group, a tridecyloxy group, a tetradecyloxy group, a pentadecyloxy group, a hexadecyloxy group, a heptadecyloxy group, an octadecyloxy group, a nonadecyloxy group, and an eicosyloxy group. Among these groups, R is preferably a straight chain alkoxy group having 1 to 5 carbon atoms or branched chain alkoxy group having 3 to 5 carbon atoms, more preferably a straight chain alkoxy group having 1 to 3 carbon atoms or branched chain alkoxy group having 3 carbon atoms, still more preferably a methoxy group or an ethoxy group, and particularly preferably a methoxy group.

Examples of the hydrophilic monomer having an alkylene glycol chain include polyethylene glycol monoacrylate; polyethylene glycol monomethacrylate; alkoxy polyethylene glycol (meth)acrylates such as methoxy polyethylene glycol acrylate, methoxy polyethylene glycol methacrylate (poly(ethylene glycol) methyl ether methacrylate, PEGMA), ethoxy polyethylene glycol acrylate, and ethoxy polyethylene glycol methacrylate; polypropylene glycol monoacrylate; polypropylene glycol monomethacrylate; alkoxy polypropylene glycol (meth)acrylates such as methoxy polypropylene glycol acrylate, methoxy polypropylene glycol methacrylate, ethoxy polypropylene glycol acrylate, and ethoxy polypropylene glycol methacrylate, phenoxy polyethylene glycol (meth)acrylate, and ethoxylated-o-phenylphenol acrylate. Among them, from the viewpoint of imparting a specific swelling property, ease of synthesis, operability, and the like, the hydrophilic monomer preferably includes at least one selected from the group consisting of polyethylene glycol monoacrylate, polyethylene glycol monomethacrylate, alkoxypolyethylene glycol monoacrylate, and alkoxypolyethylene glycol monomethacrylate, preferably includes at least one selected from the group consisting of methoxy polyethylene glycol (meth)acrylate and ethoxy polyethylene glycol (meth)acrylate, more preferably methoxy polyethylene glycol (meth)acrylate or ethoxy polyethylene glycol (meth)acrylate, and particularly preferably methoxy polyethylene glycol (meth)acrylate.

That is, in a preferred embodiment of the present disclosure, the structural unit derived from a hydrophilic monomer is a structural unit derived from at least one monomer selected from the group consisting of acrylamide, N-methyl acrylamide, N,N-dimethylacrylamide (DMAA), N-ethylacrylamide, N,N-diethylacrylamide (DEAA), N,N-dimethylaminopropylacrylamide, N,N-dimethylaminopropylmethacrylamide, 2-acrylamide-2-methylpropanesulfonic acid, N-(2-hydroxyethyl) acrylamide, N-(2-hydroxypropyl) acrylamide, N-(2-hydroxybutyl) acrylamide, polyethylene glycol monoacrylate, polyethylene glycol monomethacrylate, alkoxypolyethylene glycol monoacrylate, and alkoxypolyethylene glycol monomethacrylate. In a more preferred embodiment of the present disclosure, the structural unit is derived from at least one monomer selected from the group consisting of acrylamide, N,N-dimethylacrylamide, N,N-diethylacrylamide, methoxy polyethylene glycol (meth)acrylate, and ethoxy polyethylene glycol (meth)acrylate. In a further preferred embodiment of the present disclosure, the structural unit derived from a hydrophilic monomer is a structural unit derived from N,N-dimethylacrylamide, acrylamide, methoxy polyethylene glycol (meth)acrylate, or ethoxy polyethylene glycol (meth)acrylate. In a particularly preferred embodiment of the present disclosure, the structural unit derived from a hydrophilic monomer is a structural unit derived from N,N-dimethylacrylamide or methoxy polyethylene glycol (meth)acrylate.

In a most preferred embodiment of the present disclosure, the structural unit derived from a hydrophilic monomer is a structural unit derived from N,N-dimethylacrylamide.

The above hydrophilic monomer may be used singly or in combination of two or more kinds thereof. That is, the structural unit derived from a hydrophilic monomer (hydrophilic moiety) may be a homopolymer type composed of one kind of the hydrophilic monomer or a copolymer type composed of two or more kinds of the hydrophilic monomers. When two or more kinds of the above hydrophilic monomers are used, the form of the hydrophilic moiety may be a block copolymer, a random copolymer, or an alternating copolymer.

The structural unit having an epoxy group constituting the first copolymer is not particularly limited as long as it has an epoxy group (—C₂H₃O) but is preferably derived from a monomer having a glycidyl group (—CH₂—C₂H₃O).

In addition, the structural unit having an epoxy group preferably further has an ethylenically unsaturated group such as an acryloyl group (CH₂═CH—C(═O)—), a methacryloyl group (CH₂═C(CH₃)—C(═O)—), and a vinyl group (CH₂═CH—).

That is, in a preferred embodiment of the present disclosure, the first copolymer is a structural unit derived from a hydrophilic monomer having an amino group (—N(R1)(R2); R1 and R2 each independently represent a hydrogen atom or an unsubstituted or substituted alkyl group having 1 to 4 carbon atoms) or an alkylene glycol chain; and a structural unit having an ethylenically unsaturated group and a glycidyl group.

Specific examples of the monomer having such an epoxy group include a (meth)acrylate having a glycidyl group (epoxy group), such as glycidyl acrylate, glycidyl methacrylate (GMA), 3,4-epoxycyclohexylmethyl acrylate, 3,4-epoxycyclohexylmethyl methacrylate, β-methylglycidyl acrylate, or β-methylglycidyl methacrylate; and a vinyl ether having a glycidyl group (epoxy group), such as allyl glycidyl ether, but are not limited thereto. Among these groups, it is preferable that the structural unit having an epoxy group is a structural unit derived from at least one monomer selected from the group consisting of glycidyl acrylate, glycidyl methacrylate (GMA), 3,4-epoxycyclohexyl methyl acrylate, 3,4-epoxycyclohexyl methyl methacrylate, β-methylglycidyl acrylate, and β-methylglycidyl methacrylate from the viewpoint of further improvement in durability and easy control of polymerization of the copolymer. The structural unit having an epoxy group is more preferably a structural unit derived from at least one monomer selected from the group consisting of glycidyl acrylate and glycidyl methacrylate. The structural unit having an epoxy group is still more preferably a structural unit derived from glycidyl acrylate or glycidyl methacrylate, and particularly preferably a structural unit derived from glycidyl methacrylate. According to such a form, there is also an advantage that it is easy to form a bond with the base layer and the second layer more firmly and it is easier to manufacture medical device 100.

The monomers having an epoxy group may be used singly or in combination of two or more kinds thereof. That is, the structural unit having an epoxy group may be a homopolymer type composed of one kind of a structural unit or a copolymer type composed of two or more kinds of structural units. When two or more kinds of the above monomers having an epoxy group are used, a block copolymer, a random copolymer, or an alternating copolymer may be used.

The structure of the first copolymer is also not particularly limited, and may be any of a random copolymer, an alternating copolymer, a periodic copolymer, and a block copolymer.

Preferably, the first copolymer is a random copolymer or a block copolymer, and more preferably, the first copolymer is a block copolymer.

The composition of the structural unit of the first copolymer is appropriately selected in consideration of the durability of the lubricating layer and the desired swelling ratio of the first layer. Specifically, the composition of the structural unit derived from a hydrophilic monomer and the structural unit having an epoxy group in the first copolymer is such that the molar ratio of the structural unit derived from a hydrophilic monomer to the structural unit having an epoxy group is more than 2, preferably 3 or more, more preferably 6 or more, particularly preferably 10 or more, and most preferably 12 or more. In the composition of the structural unit derived from a hydrophilic monomer and the structural unit having an epoxy group in the first copolymer, the molar ratio of the structural unit derived from a hydrophilic monomer to the structural unit having an epoxy group is less than 100, preferably 90 or less, more preferably 80 or less, and particularly preferably 70 or less. The composition of the structural unit derived from a hydrophilic monomer and the structural unit having an epoxy group in the first copolymer (molar ratio of the structural unit derived from a hydrophilic monomer: the structural unit having an epoxy group) is more than 2:1 and less than 100:1, preferably 3:1 to 90:1 or 3:1 to 80:1, more preferably 3:1 to 70:1, still more preferably 6:1 to 70:1, particularly preferably 10:1 to 70:1, and most preferably 12:1 to 70:1. The composition of each structural unit is substantially equal to the ratio of the charged amount (mol) of the monomer to the total charged amount (mol) of each monomer when the first polymer is manufactured.

The first copolymer essentially includes the structural unit derived from a hydrophilic monomer and the structural unit having an epoxy group but may have other structural units in addition to these structural units. When the copolymer has another structural unit, examples of the monomer constituting the other structural unit include acrylic acid, methacrylic acid, acryloylmorpholine, N-vinylpyrrolidone, 2-methacryloyloxyethyl phosphorylcholine, 2-methacryloyloxyethyl-D-glycoside, 2-methacryloyloxyethyl-D-mannoside, vinyl methyl ether, 2-hydroxyethyl (meth)acrylate, 4-hydroxybutyl (meth)acrylate, 2-hydroxypropyl (meth)acrylate, 2-hydroxybutyl (meth)acrylate, 6-hydroxyhexyl(meth)acrylate, 1,4-cyclohexanedimethanol mono(meth)acrylate, 1-chloro-2-hydroxypropyl (meth)acrylate, 1,6-hexanediol mono(meth)acrylate, pentaerythritol tri(meth)acrylate, and dipentaerythritol penta(meth)acrylate, neopentyl glycol mono(meth)acrylate, trimethylolpropane di(meth)acrylate, trimethylolethane di(meth)acrylate, 2-hydroxy-3-phenyloxypropyl (meth)acrylate, 4-hydroxycyclohexyl (meth)acrylate, 2-hydroxy-3-phenyloxy (meth)acrylate, 4-hydroxycyclohexyl (meth)acrylate, cyclohexanedimethanol mono(meth)acrylate, adipic acid, glutaric acid, triethylene glycol, and tripropylene glycol. The other structural unit may be composed of only one structural unit or two or more structural units. When there is a plurality of structural units, the other structural units may exist in a block shape or in a random shape.

When the first copolymer has other structural units, the content of the other structural units is preferably more than 0 mol % and less than 10 mol % with respect to all the structural units constituting the first copolymer. More preferably, the first copolymer is substantially composed of the structural unit derived from a hydrophilic monomer and the structural unit having an epoxy group (content of other structural units=more than 0 mol % and less than 5 mol %). In this form, the first copolymer (therefore, a medical device having a first layer 102a including the copolymer) can exhibit high durability.

In an embodiment of the present disclosure, the first copolymer is substantially composed of: a structural unit derived from at least one hydrophilic monomer selected from the group consisting of (meth)acrylamide, N-methyl (meth)acrylamide, N,N-dimethyl (meth)acrylamide, N-ethyl (meth)acrylamide, N,N-diethyl (meth)acrylamide, N,N-dimethylaminopropylacrylamide, N,N-dimethylaminopropylmethacrylamide, 2-acrylamide-2-methylpropanesulfonic acid, N-(2-hydroxyethyl) acrylamide, N-(2-hydroxypropyl) acrylamide, N-(2-hydroxybutyl) acrylamide, polyethylene glycol monoacrylate, polyethylene glycol monomethacrylate, alkoxypolyethylene glycol monoacrylate, and alkoxypolyethylene glycol monomethacrylate; and a structural unit derived from a monomer having at least one epoxy group selected from the group consisting of glycidyl acrylate, glycidyl methacrylate (GMA), 3,4-epoxycyclohexyl methyl acrylate, 3,4-epoxycyclohexyl methyl methacrylate, β-methylglycidyl acrylate, and β-methylglycidyl methacrylate.

In an embodiment of the present disclosure, the first copolymer is substantially composed of a structural unit derived from at least one monomer selected from the group consisting of acrylamide, N,N-dimethyl (meth)acrylamide, N,N-diethylacrylamide, methoxy polyethylene glycol (meth)acrylate, and ethoxy polyethylene glycol (meth)acrylate, and a structural unit derived from a monomer having at least one epoxy group selected from the group consisting of glycidyl acrylate and glycidyl methacrylate.

In an embodiment of the present disclosure, the first copolymer is substantially composed of a structural unit derived from N,N-dimethyl (meth)acrylamide, acrylamide, methoxy polyethylene glycol (meth)acrylate, or ethoxy polyethylene glycol (meth)acrylate, and a structural unit derived from glycidyl acrylate or glycidyl methacrylate.

In an embodiment of the present disclosure, the first copolymer is substantially composed of a structural unit derived from N,N-dimethyl (meth)acrylamide or methoxy polyethylene glycol (meth)acrylate, and a structural unit derived from glycidyl acrylate or glycidyl methacrylate.

In an embodiment of the present disclosure, the first copolymer is substantially composed of a structural unit derived from N,N-dimethylacrylamide and a structural unit derived from glycidyl acrylate.

The terminal group of the first copolymer is not particularly limited and is appropriately defined depending on the type of raw material to be used but is usually a hydrogen atom.

The weight average molecular weight of the first copolymer is thousands to millions, preferably 10,000 to 5,000,000. In the present description, as the “weight average molecular weight”, a value measured by gel permeation chromatography (Gel Permeation Chromatography, GPC) using polystyrene as a standard substance and tetrahydrofuran (THF) as a mobile phase is adopted. The molecular weight of the copolymer can also be calculated from the kind/type of the repeating unit and the number of the repeating units.

The thickness (dry film thickness) of the first layer 102a is, for example, 0.1 to 10 μm, preferably 0.5 to 5 μm, and more preferably about 1 to 3 μm.

The method for manufacturing the first copolymer is not particularly limited, and the first copolymer can be produced by applying a conventionally known polymerization method such as a living radical polymerization method, a polymerization method using a macro initiator, or a polycondensation method.

Among these methods, the living radical polymerization method or the polymerization using a macro initiator is preferably used to arrange each structural unit in a block shape. The living radical polymerization method is not particularly limited. For example, methods described in JP H11-263819 A, JP 2002-145971 A, JP 2006-316169 A, and the like, or an atom transfer radical polymerization (ATRP) method, and the like, can be applied in the same manner or appropriately modified. In addition, in the polymerization method using a macro initiator, for example, a macro initiator having a monomer with an epoxy group and a radical polymerizable group such as a peroxide group is produced, and then the macro initiator and a hydrophilic monomer are polymerized in a polymerization solvent, whereby the first copolymer can be produced.

In the polymerization, the mixing ratio of the hydrophilic monomer and the monomer constituting the structural unit having an epoxy group is preferably controlled so as to obtain the above composition.

The polymerization solvent (solvent for polymerization) is appropriately selected from solvents in which each monomer can be dissolved. For example, water, dimethylsulfoxide, chlorobenzene, and the like are used, and dimethylsulfoxide and chlorobenzene are preferably used from the viewpoint of solubility of the monomer.

In the polymerization, the polymerization conditions are also not particularly limited as long as the copolymerization proceeds. For example, the polymerization temperature is preferably 30 to 150° C., and more preferably 40 to 100° C. The polymerization time is preferably 30 minutes to 30 hours, and more preferably 3 to 24 hours. The polymerization can be performed in an inert atmosphere such as a nitrogen atmosphere or an argon atmosphere.

Furthermore, in the manufacturing of the first copolymer, a chain transfer agent, a polymerization rate adjusting agent, a surfactant, a water-soluble polymer, a water-soluble inorganic compound (an alkali metal salt, an alkali metal hydroxide, a polyvalent metal salt, a non-reducing alkali metal salt pH buffering agent, and the like), an inorganic acid, an inorganic acid salt, an organic acid and an organic acid salt, and other additives may be appropriately used as necessary.

The first copolymer after copolymerization is preferably purified by a general purification method such as a reprecipitation method, a dialysis method, an ultrafiltration method, or an extraction method.

The first layer essentially includes the first copolymer. The first layer may include other components in addition to the first copolymer. Here, the other components are not particularly limited. For example, in a case where the medical device is intended to be inserted into a body cavity or a lumen such as a catheter, examples thereof include agents (physiologically active substances) such as an anticancer agent, an immunosuppressive agent, an antibiotic, an antirheumatic agent, an antithrombotic agent, an HMG-COA reductase inhibitor, an ACE inhibitor, a calcium antagonist, an antihyperlipidemic agent, an integrin inhibitor, an antiallergic agent, an antioxidant, a GPIIbIIIa antagonist, a retinoid, a flavonoid, a carotenoid, a lipid improver, a DNA synthesis inhibitor, a tyrosine kinase inhibitor, an antiplatelet agent, a vascular smooth muscle proliferation inhibitor, an anti-inflammatory agent, a biologically derived material, an interferon, and an NO production promoter. Here, the amounts of other components to be added are not particularly limited, and the amounts usually used can be similarly applied. Finally, the amounts of other components to be added are appropriately selected by the attending physician in consideration of the severity of the applied disease, the weight of the patient, and the like. Preferably, the first layer is substantially free of other components (that is, the first layer is substantially composed of the above first copolymer). Specifically, the content of other components is preferably less than 10% by mass (in terms of solid content), more preferably less than 5% by mass (in terms of solid content) with respect to the total mass of the first layer, and it is particularly preferable that the first layer does not include other components (that is, the first layer is composed of the first copolymer.).

### (Second Layer)

The second layer is formed on at least a part of the first layer. Preferably, the second layer is formed on the whole surface of the first layer.

The second layer includes a second copolymer having a structural unit having an alkyl vinyl ether group and a structural unit having a carboxyl group or a salt or ester thereof. Due to the presence of the second copolymer, the second layer can exhibit a lubricating property which is high (a swelling property in a wet state).

The second layer may have a one-layer form or a laminated form of two or more layers. Preferably, the second layer is in one-layer form.

The second copolymer has a structural unit having a carboxyl group or a salt or ester thereof. When the second copolymer having the structural unit comes into contact with a body fluid or blood, the second copolymer swells and gels, and can exhibit the excellent lubricating property. Therefore, the second layer including the second copolymer can exhibit excellent lubricating property. From the viewpoint of the effect of the present disclosure (particularly the lubricating property), the structural unit preferably includes at least one of a structural unit represented by the following formula (1) and a structural unit represented by the following formula (2). From the viewpoint of further improving durability and the like, it is more preferable that the structural unit includes a structural unit represented by the following formula (2). The second copolymer may have one or more of the structural unit of formula (1) and the structural unit of formula (2) in combination. The plurality of structural units may exist in a block shape or in a random shape.

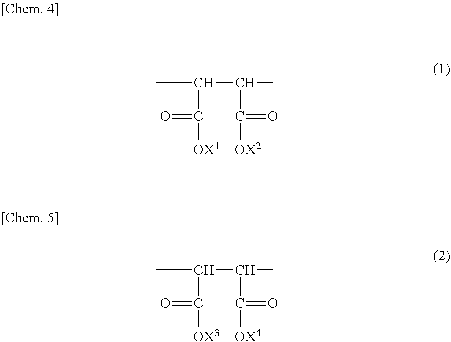

In the above formula (1), X1 represents a hydrogen atom, an alkali metal, or an alkaline earth metal. Here, examples of the alkali metal include lithium, sodium, potassium, rubidium, and cesium. Examples of the alkaline earth metal include magnesium, calcium, strontium, and barium. When X1 is an alkaline earth metal, adjacent carboxyl groups/esters are linked via X1 (—C(═O)—O—X1-O—C(═O)—). Preferably, X1 is a hydrogen atom, sodium, potassium, magnesium, or calcium, more preferably a hydrogen atom, sodium, or calcium, still more preferably a hydrogen atom or sodium (that is, a form in which at least a part of —COOX1 is converted (neutralized) to a sodium salt), and particularly preferably sodium (that is, a form in which all —COOX1 is converted (neutralized) to a sodium salt). In particular, when at least a part of X1 is sodium, the sodium salt of the carboxyl group in the second copolymer is dissociated in the body fluid or blood at the time of contact with the body fluid or blood, and the second copolymer is swollen and gelled by retaining water molecules, and particularly the excellent lubricating property can be exhibited. X2 represents a hydrogen atom, an alkali metal, an alkaline earth metal, or a straight chain alkyl group having 1 to 24 carbon atoms, or a branched chain alkyl group having 3 to 24 carbon atoms. X1 and X2 may be the same or different. When the second copolymer has the structural unit of the above formula (1), the second copolymer has the structural unit of formula (1) in which X2 is a straight chain alkyl group having 1 to 24 carbon atoms or a branched chain alkyl group having 3 to 24 carbon atoms (that is, except for the form of formula (1) in which all second copolymers are X1, X2=hydrogen atom, alkali metal, or alkaline earth metal).

Here, the alkali metal and the alkaline earth metal are the same as defined in X1 above. Examples of the alkyl group include a methyl group, an ethyl group, a propyl group, an isopropyl group, a butyl group, an isobutyl group, a sec-butyl group, a tert-butyl group, a pentyl group, an isopentyl group, a tert-pentyl group, a neopentyl group, a hexyl group, an isohexyl group, a heptyl group, an octyl group, a nonyl group, a decyl group, an undecyl group, a dodecyl group, a 2-ethylhexyl group, a tridecyl group, a tetradecyl group, a pentadecyl group, a hexadecyl group, a heptadecyl group, an octadecyl group, a nonadecyl group, an eicosyl group, a heneicosyl group, and a docosyl group. Among these groups, in consideration of the lubricating property, durability, and the like, the alkyl group as X2 is preferably a straight chain alkyl group having 1 to 8 carbon atoms or a branched chain alkyl group having 3 to 8 carbon atoms, more preferably a straight chain alkyl group having 1 to 3 carbon atoms or a branched chain alkyl group having 3 carbon atoms, particularly preferably a methyl group or an ethyl group, and most preferably, an ethyl group (that is, all —OX2 is —OC₂H₅).

That is, in a preferred embodiment of the present disclosure, the second copolymer has the structural unit represented by the above formula (1) in which X1 is a hydrogen atom, sodium, potassium, magnesium, or calcium, and X2 is a straight chain alkyl group having 1 to 8 carbon atoms or a branched chain alkyl group having 3 to 8 carbon atoms. In a more preferred embodiment of the present disclosure, the second copolymer has the structural unit represented by the above formula (1) in which X1 is a hydrogen atom, sodium or calcium, and X2 is a straight chain alkyl group having 1 to 3 carbon atoms or a branched chain alkyl group having 3 carbon atoms. In a further preferred embodiment of the present disclosure, the second copolymer has the structural unit represented by the above formula (1) in which X1 is a hydrogen atom or sodium (particularly sodium), and X2 is a methyl group or an ethyl group (particularly an ethyl group). In a particularly preferred embodiment of the present disclosure, the second copolymer is substantially composed of the structural unit represented by the above formula (1) in which X1 is sodium and X2 is an ethyl group (content of structural unit of formula (1)=more than 95 mol % and 100 mol % or less).

In formula (2), X3 represents a hydrogen atom, an alkali metal, or an alkaline earth metal. X4 represents a hydrogen atom, an alkali metal or an alkaline earth metal. X3 and X4 may be the same or different. Here, since the alkali metal and the alkaline earth metal have the same definitions as those in the above formula (1), the description thereof is not repeated here. When the second copolymer has the structural unit of the above formula (2), the structural unit of formula (2) is in a form in which at least a part of carboxyl groups (a part of —COOX3 and —COOX4) is neutralized with an alkali metal salt or alkaline earth metal salt of a carboxyl group.

Preferably, X3 is a hydrogen atom, sodium, potassium, magnesium or calcium, more preferably a hydrogen atom, sodium or calcium, and particularly preferably a hydrogen atom or sodium. X4 is a hydrogen atom, sodium, potassium, magnesium, or calcium, more preferably a hydrogen atom, sodium, or calcium, and particularly preferably a hydrogen atom or sodium. At least a part of —COOX3 and —COOX4 can be a sodium salt, a potassium salt, a magnesium salt, or a calcium salt, is preferably a sodium salt or a calcium salt, and is more preferably a sodium salt.

As described above, in the structural unit of the formula (2), at least a part of carboxyl groups (at least a part of —COOX3 and —COOX4) are an alkali metal salt or an alkaline earth metal salt of carboxyl groups, and the proportion (%) of an alkali metal salt or an alkaline earth metal salt of a carboxyl group to all carboxyl groups (salts) present in the structural unit of formula (2) constituting the second copolymer is preferably 10 to 100%, more preferably more than 10% and not more than 100%, even more preferably 25 to 100%, particularly preferably more than 50% and 100% or less, and most preferably 60 to 100%. In an alkali metal salt or an alkaline earth metal salt of a carboxyl group, —COOX, X denotes an alkali metal or an alkaline earth metal. The proportion (%) of an alkali metal salt or an alkaline earth metal salt of a carboxyl group to all carboxyl groups (salts) present in the structural unit of formula (2) constituting the second copolymer is calculated as: (number of —COOX)×100/[(number of —COOX3)+ (number of —COOX4)].

In the present description, the proportion (%) of an alkali metal salt or an alkaline earth metal salt of a carboxyl group to all carboxyl groups (salts) present in the structural unit of formula (2) constituting the second copolymer (—COOX; X=an alkali metal or an alkaline earth metal) can also be simply referred to as the “alkali (earth) metal salt percentage (%)”.

That is, in a preferred embodiment of the present disclosure, the second copolymer has the structural unit represented by the above formula (2) in which X3 is a hydrogen atom, sodium, potassium, magnesium or calcium, X4 is a hydrogen atom, sodium, potassium, magnesium or calcium, and the alkali (earth) metal salt percentage (%) is 10 to 100% (i.e., more than 10% and 100% or less). In a more preferred embodiment of the present disclosure, the second copolymer has the structural unit represented by the above formula (2) in which X3 is a hydrogen atom, sodium or calcium, X4 is a hydrogen atom, sodium or calcium, and the alkali (earth) metal salt percentage (%) is 25 to 100% (i.e., more than 50% and 100% or less). In a particularly preferred embodiment of the present disclosure, the second copolymer has the structural unit represented by the above formula (2) in which X3 is a hydrogen atom or sodium, X4 is a hydrogen atom or sodium, and the alkali (earth) metal salt percentage (%) is 60 to 100% (i.e., equal to or more than 60% and 100% or less).

The second copolymer further has the structural unit having an alkyl vinyl ether group. From the viewpoint of the effect of the present disclosure (particularly durability), the structural unit having an alkyl vinyl ether group is preferably represented by the following formula (3).

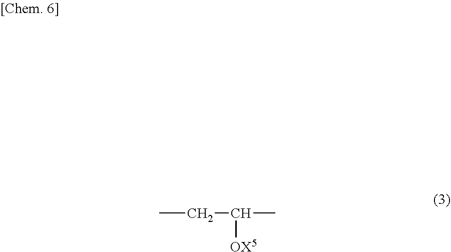

In formula (3), X5 is a straight chain alkyl group having 1 to 24 carbon atoms or a branched chain alkyl group having 3 to 24 carbon atoms. Here, since the alkyl group has the same definitions as those in the above formula (1), the description thereof is not repeated here. Among these groups, in consideration of the lubricating property, durability, and the like, X5 is preferably a straight chain alkyl group having 1 to 5 carbon atoms or a branched chain alkyl group having 3 to 5 carbon atoms, more preferably a straight chain or branched chain alkyl group having 1 to 3 carbon atoms, still more preferably a methyl group or an ethyl group, and particularly preferably a methyl group. That is, the structural unit having an alkyl vinyl ether group is particularly preferably a methyl vinyl ether group (—CH₂—CH(OCH₃)—) (i.e., the methyl vinyl ether group, —CH₂—CH(OCH₃)—, forms the backbone of the second copolymer).

The structure of the second copolymer is also not particularly limited, and may be any of a random copolymer, an alternating copolymer, a periodic copolymer, and a block copolymer. Preferably, the second copolymer is an alternating copolymer of the structural unit of the above formula (1) and/or (2) and the structural unit of the above formula (3).

The composition of the structural unit of the second copolymer is appropriately selected in consideration of the lubricating property of the lubricating layer and the like. Specifically, the composition of the structural unit having an alkyl vinyl ether group and the structural unit position having a carboxyl group or a salt or ester thereof in the second copolymer (molar ratio of structural unit having alkyl vinyl ether group: structural unit having carboxyl group or salt or ester thereof) is preferably 0.5:1 or more and 2:1 or less, more preferably 0.75:1 or more and 1.3:1 or less, and particularly preferably 1:1. The composition of each structural unit is substantially equal to the molar ratio of the charged amount (in mol) of the monomer to the total charged amount (in mol) of each monomer when the second polymer is manufactured.

The second copolymer essentially includes the structural unit having an alkyl vinyl ether group and the structural unit having a carboxyl group or a salt or ester thereof but may have other structural units in addition to these structural units. When the copolymer has another structural unit, the monomer constituting the other structural unit has the same definition as that of the first copolymer, and thus the description thereof is not repeated here.

When the second copolymer has other structural units, the content of the other structural units is preferably more than 0 mol % and less than 10 mol % with respect to all the structural units constituting the second copolymer. More preferably, the second copolymer is substantially composed of the structural unit having an alkyl vinyl ether group and the structural unit having a carboxyl group or a salt or ester thereof (content of other structural units=more than 0 mol % and less than 5 mol %). Particularly preferably, the second copolymer is composed of the structural unit having an alkyl vinyl ether group and the structural unit having a carboxyl group or a salt or ester thereof (the content of other structural units=0 mol %). In this form, the second copolymer (Therefore, a medical device having a second layer including the copolymer) can exhibit high durability (i.e., a lubrication-retaining property).

The second copolymer has the structural unit of formula (3) and at least one of the structural unit of formula (1) and the structural unit of formula (2). Preferably, the second copolymer has the structural unit of formula (3) and only one of the structural unit of formula (1) and the structural unit of formula (2). More preferably, the second copolymer is composed of the structural unit of formula (3) and one of the structural unit of formula (1) and the structural unit of formula (2) (i.e., the second copolymer is composed of the structural unit of formula (1) and the structural unit of formula (3), or composed of the structural unit of formula (2) and the structural unit of formula (3)). Particularly preferably, the second copolymer is composed of both the structural unit of formula (2) and the structural unit of formula (3).

The terminal group of the second copolymer is not particularly limited and is appropriately defined depending on the type of raw material to be used, but is usually a hydrogen atom.

The molecular weight of the second copolymer is also not particularly limited and is appropriately selected in consideration of a desired effect (for example, the lubricating property and durability (lubrication retaining property) in a wet state) and the like. Specifically, the weight average molecular weight of the second copolymer is preferably 10,000 to 7,000,000, and more preferably 100,000 to 5,000,000.

The thickness (dry film thickness) of the second layer is, for example, 0.1 to 10 μm, preferably 0.5 to 5 μm, and more preferably about 0.5 to 2 μm.

In addition, the method for manufacturing the second copolymer is not particularly limited, and for example, when the second copolymer has the structural unit of formula (1) and the structural unit of formula (3), the method can be applied in the same manner as the known method described in WO 2015/029625 and the like or after being appropriately modified. For example, the second copolymer can be manufactured by reacting a maleic anhydride-based polymer represented by the following formula (4):

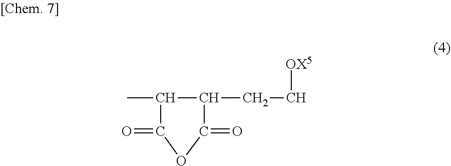

(in formula (4), X5 has the same definition as that of X5 in formula (3)) with a mixed solution including a straight chain alcohol having 1 to 24 carbon atoms or branched chain alcohol having 3 to 24 carbon atoms and water (hereinafter, it can also be simply referred to as a mixed solution) to simultaneously perform an esterification reaction and a hydrolysis reaction of the maleic anhydride-based polymer to obtain an esterified maleic acid-based polymer, and then subjecting the esterified maleic acid-based polymer to an alkali treatment. Here, for the maleic anhydride-based polymer, a commercially available product may be used, and examples thereof include GANTREZ AN series manufactured by Ashland Japan Co., Ltd. The content mass ratio of alcohol:water in the mixed solution is preferably 100:0.1 to 50, and more preferably 100:0.5 to 10. The maleic anhydride is completely ring-opened, and esterification with alcohol tends to proceed completely by using a solution in which the content mass ratio of alcohol:water is within the above range. For the straight chain alcohol having 1 to 24 carbon atoms or the branched chain alcohol having 3 to 24 carbon atoms used for esterification, an alcohol corresponding to X2 in the above formula (1) may be used. The mass ratio between the maleic anhydride-based polymer and the mixed solution used in the reaction may be appropriately set so that the reaction proceeds, but usually, the mass ratio is maleic anhydride-based polymer:mixed solution=100:50 to 5000 (mass ratio), preferably maleic anhydride-based polymer:mixed solution=about 100:500 to 1000 (mass ratio). The esterification reaction and the hydrolysis reaction are performed by heating and refluxing the maleic anhydride-based polymer using a mixed solution of alcohol and water. The reflux condition is appropriately selected depending on the solvent (alcohol) to be used. In order to allow the reaction to proceed sufficiently, for example, the reflux time is preferably 5 to 120 hours. The temperature at the time of reflux is appropriately set depending on the type of alcohol to be used and the content mass ratio of alcohol and water, but is preferably about 50 to 100° C.

After the esterification reaction and the hydrolysis reaction, an alkali treatment is performed. If necessary, purification may be performed by a general purification method such as a reprecipitation method, a dialysis method, an ultrafiltration method, or an extraction method after the esterification reaction and the hydrolysis reaction and before the alkali treatment. In addition, the esterified maleic acid-based polymer may be subjected to alkali treatment in the form of a solid or in the form of a solution.

At least a part of the carboxyl group (—COOH) and the ester moiety (—COOX2) in the esterified maleic acid-based polymer is converted to a carboxylate (—COOX; X=alkali metal or alkaline earth metal) by the alkali treatment. When the alkali-treated second copolymer comes into contact with a body fluid or blood, the carboxylate in the second copolymer swells and becomes gelled due to the body fluid or blood, so that an excellent lubricating property can be exhibited. In the alkali treatment, the alkali compound used for preparing the alkali solution is not particularly limited, and examples thereof include sodium hydroxide, potassium hydroxide, sodium carbonate, potassium carbonate, calcium hydroxide, sodium hydrogen carbonate, potassium hydrogen carbonate, lithium hydroxide, sodium, and ammonia. Among these alkali compounds, sodium hydrogen carbonate, sodium hydroxide, potassium hydroxide, sodium carbonate, and calcium hydroxide are preferable, sodium hydrogen carbonate, sodium hydroxide, and sodium carbonate are more preferable, and sodium hydrogen carbonate is particularly preferable. The amount of the alkali compound to be added can be appropriately selected according to the alkali (earth) metal salt percentage (%). In the repeating unit of the esterified maleic acid-based polymer, a carboxyl group (—COOH) and an ester moiety (—COOX2) exist. Therefore, for example, in order to convert (neutralize) all the carboxyl groups in the esterified maleic acid-based polymer (degree of esterification=50%) into an alkali metal salt, 1 mol equivalent of an alkali compound is added to the repeating unit of the esterified maleic acid-based polymer. For example, in order to convert (neutralize) 60% of the carboxyl groups in the esterified maleic acid-based polymer (degree of esterification=50%) into an alkali metal salt, 0.6 mol equivalent of an alkali compound is added to the repeating unit of the esterified maleic acid-based polymer. The solvent for dissolving the alkali compound is not particularly limited, and examples thereof include water, alcohols such as methanol, ethanol, isopropanol, and ethylene glycol, ketones such as acetone and methyl ethyl ketone, esters such as ethyl acetate, halides such as chloroform, alkanes such as butane and hexane, ethers such as tetrahydrofuran and butyl ether, aromatics such as benzene and toluene, and amides such as N,N-dimethylformamide (DMF).

The above solvent may be used singly or in combination of two or more kinds thereof. The alkaline solution may include other components in addition to the alkaline compound. Here, examples of other components include sodium chloride, sodium bromide, potassium chloride, potassium bromide, lithium chloride, and lithium bromide. Sodium chloride is preferred. A preferred concentration of the other component is not particularly limited and can be in any amount so as to have a concentration of preferably 0.01 to 10% by mass in the alkaline solution. The alkali treatment conditions are not particularly limited. For example, the alkali treatment temperature is preferably 15 to 50° C., and more preferably 20 to 30° C. The alkali treatment time is preferably 5 minutes to 3 hours, and more preferably 15 minutes to 1 hour. If necessary, the alkali treatment may be performed while stirring. The liquid obtained by the alkali treatment may be used as it is as a second coating liquid described in detail below. Alternatively, after the alkali treatment, a washing step may be performed if necessary. The washing step can easily complete the conversion of the carboxyl group (—COOH) and the ester moiety (—COOX2) to carboxylate (—COOX; X=alkali metal or alkaline earth metal) by the alkali treatment. Here, the washing condition is not particularly limited. Examples of the washing liquid (solvent) that can be used in the washing step include water, alcohols such as methanol, ethanol, isopropanol, and ethylene glycol, ketones such as acetone and methyl ethyl ketone, esters such as ethyl acetate, halides such as chloroform, alkanes such as butane and hexane, ethers such as tetrahydrofuran and butyl ether, aromatics such as benzene and toluene, and amides such as N,N-dimethylformamide (DMF). The above solvent may be used singly or in combination of two or more kinds thereof. The temperature of the washing liquid is preferably 0 to 70° C., and more preferably 20 to 65° C. The washing time is 0.1 to 120 minutes, and more preferably 0.5 to 120 minutes.

Further, for example, when the second copolymer has the structural unit of formula (2) and the structural unit of formula (3), for example, a maleic acid-based polymer represented by the following formula (5):

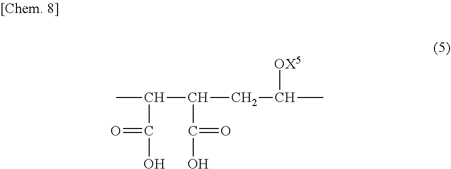

(in formula (5), X5 has the same definition as that of X5 in formula (3)) is subjected to alkali treatment. At least a part of the carboxyl group (—COOH) of the maleic acid-based polymer is converted to a carboxylate (—COOX; X=alkali metal or alkaline earth metal) by the alkali treatment to manufacture a second copolymer. When the second copolymer comes into contact with a body fluid or blood by the alkali treatment, the carboxylate in the second copolymer swells and is gelled due to the body fluid or blood, so that the excellent lubricating property can be exhibited. Here, as the maleic acid-based polymer, a commercially available product may be used, and examples thereof include poly(methyl vinyl ether-alt-maleic acid) from Sigma-Aldrich. The alkali treatment can be performed in the alkali treatment described in the case where the second copolymer has the structural unit of formula (1) and the structural unit of formula (3) except for the amount of the alkali compound to be added, and thus the description thereof is not repeated here. The amount of the alkali compound to be added can be appropriately selected according to the desired alkali (earth) metal salt percentage (%) as described above. Specifically, two carboxyl groups (—COOH) exist in the repeating unit of the esterified maleic acid-based polymer.

Therefore, for example, in order to convert (neutralize) all the carboxyl groups in the esterified maleic acid-based polymer into alkali metal salts (alkali (earth) metal salt percentage (%)=100%), 2 mol equivalents (1 mol equivalent with respect to each carboxyl group) of the alkali compound is added to the repeating unit of the esterified maleic acid-based polymer. For example, in order to convert (neutralize) 60% of the carboxyl groups in the esterified maleic acid-based polymer into an alkali metal salt (alkali (earth) metal salt percentage (%)=60%), 1.2 mol equivalent of an alkali compound is added with respect to the repeating unit of the esterified maleic acid-based polymer (amount of addition of sodium hydrogen carbonate with respect to each carboxyl group=0.6 mol equivalent).

The second layer essentially includes the second copolymer. The second layer may include other components in addition to the second copolymer. Here, the other components are not particularly limited, and since they have the same definition as that of the first layer, the description thereof is not repeated here. Preferably, the second layer is substantially free of other components (that is, the second layer is substantially composed of the second copolymer). Specifically, the content of other components is preferably less than 10% by mass (in terms of solid content), more preferably less than 5% by mass (in terms of solid content) with respect to the total mass of the second layer. It is particularly preferable that the second layer does not include other components (that is, the second layer is composed of the second copolymer).

### (Method for Manufacturing Medical Device)

A method for manufacturing a medical device according to the present disclosure includes coating a solution (first coating liquid) including a first copolymer and a solvent onto a base layer 101 to form a first precursor layer on the base layer ((I) first solution coating step); coating a solution (second coating liquid) including a second copolymer and a solvent onto the first precursor layer to form a second precursor layer on the first precursor layer, thereby obtaining an intermediate laminated body ((II) second solution coating step); and irradiating the intermediate laminated body with an active energy ray ((III) immobilization treatment step). Washing may be performed after the above steps (I) and/or (II) if necessary.

The present disclosure also provides a method for manufacturing a medical device, the method including: applying a first coating liquid including a first copolymer having a structural unit derived from a hydrophilic monomer and a structural unit having an epoxy group and a solvent to at least a part of a base layer 101 to form a first precursor layer on at least a part of the base layer; applying a second coating liquid including a second copolymer having a structural unit having an alkyl vinyl ether group and a structural unit having a carboxyl group or a salt or ester thereof and a solvent to at least a part of the first precursor layer to obtain an intermediate laminated body in which a second precursor layer is formed on at least a part of the first precursor layer; and irradiating the intermediate laminated body with an active energy ray.

Each step will be described below. The present disclosure can be applied in the same manner as a known method for manufacturing a medical device or by appropriately modifying the method except for using the first and second copolymers according to the present disclosure and is not limited to the following forms.

### (I) First Solution Coating Step

In this step, a solution including the first copolymer, a solvent, and other components as necessary (in the present description, it can also be simply referred to as a “first coating liquid”) is prepared, and the first coating liquid is applied onto the base layer 101 to form the first precursor layer on the base layer 101. The method for applying the solution is not particularly limited as long as the solution including the first copolymer and the solvent is used, and the method can be applied in the same manner as the known method or by appropriately modifying the method. When the coating liquid includes other components, the other components are the same as those described above, and thus the description thereof will not be repeated here.

The solvent used for preparing the first coating liquid is not particularly limited and is appropriately selected according to the type of the first copolymer (and other components, if used.).

From the viewpoint of high solubility, water, acetone, ethanol, methanol, dichloromethane, chloroform, carbon tetrachloride, tetrahydrofuran (THF), dimethyl sulfoxide, N,N-dimethylformamide, methyl ethyl ketone, methyl isobutyl ketone, cyclohexanone, and the like are preferably used. The above solvent may be used singly or in combination of two or more kinds thereof.

The concentration of the first copolymer in the first coating liquid is not particularly limited. For example, the concentration of the first copolymer in the first coating liquid is preferably 0.01 to 20% by mass, more preferably 0.05 to 15% by mass, and particularly preferably 1 to 10% by mass. When the concentration of the first copolymer is within the above range, the obtained first layer 102a can sufficiently exhibit the effect of the present disclosure. In addition, a uniform lubricating layer 102 having a desired thickness can be easily obtained by one coating, and the viscosity of the solution falls within an appropriate range, which is preferable in terms of operability (ease of coating, for example) and production efficiency. However, even if it is out of the above range, it can be sufficiently used as long as it does not affect the operation and effect of the present disclosure.

Next, the first coating liquid prepared above is coated onto the base layer 101. Here, since the base layer is the same as described above, the description thereof is not repeated here.

The method for applying the copolymer solution (coating liquid) to the surface of the base layer is not particularly limited, and conventionally known methods such as a coating/printing method, an immersion method (dipping method, dip coating method), a spraying method, a spin coating method, a mixed solution impregnation sponge coating method, a bar coating method (for example, the wire bar method), a die coating method, a reverse coating method, a comma coating method, a gravure coating method, and a doctor knife method can be applied. Among them, an immersion method (dipping method, dip coating method), a spraying method, and a bar coating method are preferably used.

When the first precursor layer (therefore, the first layer; the same applies hereinafter) is formed on a thin and narrow inner surface of a catheter, injection needle, or the like, the base layer 101 may be immersed in the first coating liquid and the pressure in the system may be reduced to defoam. The solution can be quickly penetrated into the thin and narrow inner surface, promoting the formation of the first precursor layer by reducing the pressure to defoam.

When the first precursor layer is formed only on a part of the base layer 101, the first precursor layer can be formed on a desired surface portion of the base layer 101 by immersing only a part of the base layer in the first coating liquid to coat a part of the base layer with the first coating liquid.

In a case where it is difficult to immerse only a part of the base layer 101 in the first coating liquid, a surface portion of the base layer on which the first precursor layer does not need to be formed is protected (covered or the like) in advance with an appropriate member or material that can be attached/detached (donned/doffed), the base layer 101 is immersed in the first coating liquid to coat the base layer 101 with the first coating liquid, and then a protective member (material) of the surface portion of the base layer 101 on which the first layer 102a does not need to be formed is removed, whereby the first precursor layer can be formed on a desired surface portion of the base layer 101. The present disclosure is not limited to these forming methods, and the first precursor layer can be formed by appropriately using a conventionally known method. For example, in a case where it is difficult to immerse only a part of the base layer 101 in the first coating liquid, another coating method (for example, a method of coating a first coating liquid to a predetermined surface portion of a medical device using a coating device such as a spray device, a bar coater, a die coater, a reverse coater, a comma coater, a gravure coater, a spray coater, or a doctor knife, or the like) may be applied instead of the immersion method. In a case where both the outer surface and the inner surface of the cylindrical tool need to have the first layer 102a due to the structure of the medical device, the immersion method (dipping method) is preferably used because both the outer surface and the inner surface can be coated at a time.

The application amount of the first coating liquid is preferably selected so that the thickness (dry film thickness) of the first layer 102a to be obtained falls within the above range.

Next, the first precursor layer formed as described above is dried if necessary to form the first precursor layer on the base layer 101. As a result, the epoxy groups of the first copolymer react with each other, and the film strength of the first layer 102a can be increased. In the present description, “supporting” means a state in which the first precursor layer is immobilized in a state of not being easily released from the surface of the base layer 101, and includes not only a form in which the entire surface of the base layer 101 is completely covered with the first precursor layer, but also a form in which only a part of the surface of the base material is covered with the first precursor layer, that is, a form in which the first precursor layer is attached to only a part of the surface of the base material.

The drying treatment includes natural drying or heat treatment. In particular, when a base material having low heat resistance is used, the drying treatment is preferably natural drying. In the case where heat treatment is applied, the conditions for the heat treatment are not particularly limited as long as the solvent can be removed (the first precursor layer can be formed on the base layer 101) and can be appropriately selected according to the type of solvent. For example, the heat treatment temperature is preferably 40 to 200° C., and more preferably 50 to 150° C. The heat treatment time is preferably 1 minute to 30 hours, more preferably 30 minutes to 15 hours, and particularly preferably 1 to 10 hours. Given the above conditions, the solvent can be efficiently removed to form the first precursor layer on the base layer.

In addition, the pressure condition in the drying treatment step is not limited at all, and the drying treatment may be performed under normal pressure (atmospheric pressure) or may be performed under increased or reduced pressure. As the drying (or heating) means (device), for example, an oven, a vacuum dryer, or the like can be used, but in the case of natural drying, the drying means (device) is particularly unnecessary.

In this step, the first precursor layer may be washed if necessary. Here, the washing method is not particularly limited, but a method of immersing the first precursor layer in a washing solvent, a method of flowing the washing solvent onto the first precursor layer, or a combination thereof may be used. The washing solvent used at this time is not particularly limited as long as it does not dissolve the first precursor layer, but water or warm water is preferably used. The temperature of the washing water is not particularly limited, but is preferably 20° C. to 100° C., and more preferably 25 to 80° C. The washing time (time for bringing the washing solvent into contact with the first precursor layer) is not particularly limited, but is preferably 1 to 60 minutes, and more preferably 5 to 30 minutes. After the washing step, a drying step may be further performed. The drying method and the drying conditions (temperature, time, etc.) are not particularly limited, and conventionally known methods can be used.

### (II) Second Solution Coating Step

In this step, a solution including the second copolymer, a solvent, and other components as necessary (in the present description, it is also simply referred to as a “second coating liquid”) is prepared, and the second coating liquid is applied onto the first precursor layer to form the second precursor layer on the first precursor layer. As a result, an intermediate laminated body in which the first precursor layer and the second precursor layer are sequentially formed on the base layer 101 is obtained.

This step can be applied by replacing the first coating liquid with the second coating liquid in the above step (I). The solvent used for preparing the second coating liquid is preferably a solvent capable of further swelling the first precursor layer. In this case, the first precursor layer is swollen by the solvent of the second coating liquid, and the second copolymer penetrates into the first precursor layer (first layer) swollen together with the solvent. Therefore, the first copolymer and the second copolymer are closer to each other. Therefore, in the next step (III), the first copolymer and the second copolymer react at more reaction points, and the second layer 102b can be more firmly bonded (crosslinked) to the first layer 102a. Therefore, the medical device of the present disclosure is further excellent in durability and can further retain the lubricating property even after repeated sliding. From the above viewpoint, the solvent used for preparing the second coating liquid is preferably an aqueous solvent (water alone or a combination of water and a water-soluble organic solvent). Examples of the water-soluble organic solvent include a lower alcohol (methanol, ethanol, propanol, isopropanol), acetone, N,N-dimethylformamide, acetonitrile, and acetic acid.

The water-soluble organic solvent may be used singly or in the form of a mixture of two or more kinds thereof. As the solvent used for preparing the second coating liquid, water, methanol, ethanol, acetone, or a mixed liquid thereof is preferably used, water, ethanol, acetone, or a mixed liquid thereof is more preferably used, and a mixed liquid of water and ethanol and a mixed liquid of water and acetone are particularly preferably used.

The application amount of the second coating liquid is preferably selected so that the thickness (dry film thickness) of the obtained second layer falls within the above range.

### (III) Immobilization Treatment Step

In this step, the intermediate laminated body obtained in the step (II) is irradiated with an active energy ray. The epoxy group of the first copolymer is cleaved to generate a reaction point by irradiation with an active energy ray, and the second copolymer and the material constituting the base layer react (chemically bond, crosslink) with the first copolymer at this reaction point. Therefore, the second layer firmly adheres to the first layer 102a and the base layer 101. The cleaved epoxy groups in the first copolymer react (chemically bond, crosslink) with each other by irradiation with an active energy ray. Therefore, the film strength of the first layer 102a is increased. Therefore, the medical device having the lubricating layer 102 according to the present disclosure can exhibit excellent durability. In addition, the active energy ray irradiation is performed at room temperature, and heating is not required. Therefore, it can be suitably used even for a base material having low heat resistance. In addition, since it is not necessary to use an initiator, it is very preferable in terms of safety.

That is, in an embodiment of the present disclosure, the lubricating layer 102 has a region where the first copolymer and the second copolymer are bonded to each other.

Examples of the active energy ray include an ultraviolet ray (UV), an electron beam, a gamma ray, and the like, and an ultraviolet ray or an electron beam are preferable, and an electron beam is more preferable. According to the electron beam irradiation, the first copolymer and the second copolymer, and the first copolymer and the material forming the base layer 101 chemically react more strongly, so that the lubricating layer 102 of the medical device can exhibit higher durability. In addition, electron beam irradiation is preferable from the viewpoint of productivity, mass production, and the like because a desired reaction (crosslinking) can be performed in a short time.

That is, the present disclosure also provides a method for manufacturing a medical device, the method including: applying a first coating liquid including a first copolymer having a structural unit derived from a hydrophilic monomer and a structural unit having an epoxy group and a solvent to at least a part of a base layer 101 to form a first precursor layer on at least a part of the base layer; applying a second coating liquid including a second copolymer having a structural unit having an alkyl vinyl ether group and a structural unit having a carboxyl group or a salt or ester thereof and a solvent to at least a part of the first precursor layer to obtain an intermediate laminated body in which a second precursor layer is formed on at least a part of the first precursor layer; and irradiating the intermediate laminated body with an electron beam.

When the active energy ray is an electron beam, the irradiation condition is appropriately selected according to the desired reactivity (therefore, the durability of the lubricating layer) between the first copolymer and the second copolymer and the material forming the base layer 101, and is selected in consideration of, for example, the type of the first copolymer or the second copolymer. For example, the irradiation temperature is preferably 10 to 80° C., more preferably 20 to 40° C. The acceleration voltage is preferably 50 to 200 kV, more preferably 50 to 70 kV. The irradiation dose is preferably 10 to 1000 kGy, more preferably 30 to 500 kGy, still more preferably 30 kGy or more and less than 500 kGy, particularly preferably 30 to 350 kGy, and most preferably 100 to 200 kGy. Given such conditions, the first copolymer and the second copolymer react more efficiently, and thus the lubricating layer can exhibit more excellent durability.

In a preferred embodiment of the present disclosure, the intermediate laminated body is irradiated with an electron beam at an irradiation dose of 30 to 500 kGy. In a preferred embodiment of the present disclosure, the intermediate laminated body is irradiated with an electron beam at an irradiation dose of 30 kGy or more and less than 500 kGy. In a preferred embodiment of the present disclosure, the intermediate laminated body is irradiated with an electron beam at an irradiation dose of 30 to 350 kGy. In a preferred embodiment of the present disclosure, the intermediate laminated body is irradiated with an electron beam at an irradiation dose of 100 to 200 kGy.

A medical device including a lubricating layer having an excellent lubricating property and durability is manufactured by the above method.

### [Application of Medical Device]

The medical device having the lubricating layer according to the present disclosure has excellent lubricating property and durability. The medical device according to the present disclosure is used in contact with a body fluid, blood, or the like, and has the lubricating property on a surface in an aqueous liquid such as a body fluid or physiological saline and can improve operability and reduce damage to mucosal tissue. Specific examples of the medical device include a catheter, a stent, and a guide wire used in a blood vessel. That is, the medical device according to an embodiment of the present disclosure is a catheter, a stent, or a guide wire. In addition, the following medical devices are shown:
(a) Catheters inserted into or indwelled in a digestive organ orally or nasally, such as a gastric tube catheter, a feeding catheter, or a feeding tube;
(b) Catheters inserted or indwelled into or in the airway or trachea orally or nasally, such as an oxygen catheter, an oxygen cannula, a tube or cuff of an endotracheal tube, a tube or cuff of a tracheostomy tube, and an intratracheal aspiration catheter;
(c) Catheters inserted into or indwelled in the urethra or ureter, such as a catheter or a balloon of a urethral tube, a urinary catheter, and a urinary balloon catheter;
(d) Catheters inserted into or indwelled in various body cavities, organs, and tissues such as a suction catheter, a drainage catheter, and a rectal catheter;
(e) Catheters inserted into or indwelled in a blood vessel, such as an indwelling needle, an IVH catheter, a thermolysion catheter, an angiographic catheter, a vasodilator catheter, a dilator, or an introducer, or a guide wire, a stylet, or the like for these catheters;
(f) Artificial trachea, artificial bronchus, etc.; and
(g) Medical devices for extracorporeal circulation treatment (an artificial lung, an artificial heart, an artificial kidney, etc.) and circuits thereof.

### EXAMPLES

Effects associated with the present disclosure will be described with reference to the following Examples and Comparative Examples. The technical scope of the present disclosure is not limited only to the following Examples. In the following examples, unless otherwise specified, operations were performed under conditions of room temperature (25° C.)/relative humidity of 40% RH or more and 50% RH or less. In addition, unless otherwise specified, “%” and “parts” mean “% by mass” and “parts by mass”, respectively.

### Synthesis Example 1: Preparation of Block Copolymer 1

To 72.3 g of adipic acid dichloride, 29.7 g of triethylene glycol was added dropwise at 50° C., and then 4.5 g of methyl ethyl ketone was added to 22.5 g of the oligoester obtained by removing hydrochloric acid under reduced pressure at 50° C. for 3 hours. The mixture was added dropwise to a solution containing 5 g of sodium hydroxide, 6.93 g of 31% hydrogen peroxide, 0.44 g of a surfactant dioctyl phosphate and 120 g of water, and reacted at −5° C. for 20 minutes. The resulting product was repeatedly washed with water and methanol, and then, dried to obtain a poly peroxide (PPO) having a plurality of peroxide groups in a molecule. Subsequently, 0.5 g of PPO as a polymerization initiator and 9.5 g of glycidyl methacrylate (GMA) were polymerized with benzene as a solvent at 65° C. for 2 hours under reduced pressure with stirring.

The reaction product was reprecipitated with diethyl ether to obtain poly-GMA (PPO-GMA) having a peroxide group in the molecule.

Subsequently, 1.35 g (corresponding to 9.5 mmol of GMA) of the obtained PPO-GMA as a polymerization initiator was dissolved in chlorobenzene together with 11.2 g (113 mmol) of N,N-dimethylacrylamide (DMAA) so as to be 1.35% by mass (PPO-GMA concentration) and 11.2% by mass (DMAA concentration), respectively, and the mixture was polymerized by heating to 80° C. for 7 hours under a nitrogen atmosphere. The reaction product was reprecipitated with cyclohexane and recovered to produce a block copolymer 1 having a structural unit derived from DMAA and a structural unit derived from GMA. The DMAA:GMA ratio of the produced the block copolymer 1 was measured by 1H-NMR, and the ratio of DMAA:GMA was 12:1 (molar ratio). Hereinafter, the block copolymer 1 is also referred to as “p(DMAA-GMA) (12/1)”.

### Synthesis Examples 2 to 7: Preparation of Block Copolymers 2 to 7

Block copolymers 2 to 7 having the structural unit derived from DMAA and the structural unit derived from GMA was produced in the same manner as in Synthesis Example 1 except that PPO-GMA and N,N-dimethylacrylamide (DMAA) were dissolved in chlorobenzene so that the ratio of DMAA:GMA was 2:1 (molar ratio), 3:1 (molar ratio), 6:1 (molar ratio), 35:1 (molar ratio), 70:1 (molar ratio), and 100:1 (molar ratio) in Synthesis Example 1. The DMAA:GMA ratio of the obtained block copolymers 2 to 7 was measured by 1H-NMR, and it was confirmed that the ratio of DMAA:GMA was 2:1 (molar ratio) (block copolymer 2), 3:1 (molar ratio) (block copolymer 3), 6:1 (molar ratio) (block copolymer 4), 35:1 (molar ratio) (block copolymer 5), 70:1 (molar ratio) (block copolymer 6), and 100:1 (molar ratio) (block copolymer 7). Hereinafter, the block copolymer 2 to 7 is also referred to as “p(DMAA-GMA) (2/1)”, “p(DMAA-GMA) (3/1)”, “p(DMAA-GMA) (6/1)”, “p(DMAA-GMA) (35/1)”, “p(DMAA-GMA) (70/1)”, and “p(DMAA-GMA) (100/1)”, respectively.

### Synthesis Example 8: Preparation of Block Copolymer 8

A block copolymer 8 having a structural unit derived from PEGMA and the structural unit derived from GMA was produced in the same manner as in Synthesis Example 1 except for using 56.0 g (113 mmol) of methoxypolyethylene glycol (n=9) methacrylate (PEGMA) (product name: M90G, manufactured by SHIN-NAKAMURA CHEMICAL Co., Ltd.) in place of N,N-dimethylacrylamide (DMAA) in Synthesis Example 1. The ratio of PEGMA:GMA in the produced block copolymer 8 was measured by 1H-NMR, and the result showed that the ratio of PEGMA:GMA was 12:1 (molar ratio). Hereinafter, the block copolymer 8 is also referred to as “p(PEGMA-GMA) (12/1)”.

### Synthesis Example 9: Preparation of Block Copolymer 9

A block copolymer 8 having a structural unit derived from 2-MEA and a structural unit derived from GMA was produced in the same manner as in Synthesis Example 1 except for using 14.7 g (113 mmol) of 2-methoxyethyl acrylate (2-MEA) in place of N,N-dimethylacrylamide (DMAA) in Synthesis Example 1. The 2-MEA:GMA ratio in produced block copolymer 9 was measured by 1H-NMR, and the result showed that the ratio of 2-MEA:GMA was 12:1 (molar ratio). Hereinafter, the block copolymer 9 is also referred to as “p (MEA-GMA) (12/1)”.

The swelling ratio of block copolymers 1 to 9 obtained in Synthesis Examples 1 to 9, poly(N,N-dimethylacrylamide) (manufactured by Scientific Polymer, weight-average molecular weight (Mw)=100,000) (pDMAA), polyvinylpyrrolidone (manufactured by Tokyo Chemical Industry Co., Ltd., K90, weight-average molecular weight=360,000) (PVP), and polyethylene glycol (manufactured by Wako Pure Chemical Industries, Ltd., average molecular weight=3,600,000 to 4,000,000) (PEG) was measured according to the above method.

The results are shown in Table 1 below.

### Example 1

To prepare a first precursor layer forming liquid, p(DMAA-GMA) (3/1) (block copolymer 3) was dissolved in acetone to be 6% by mass.

In 27.6 g of ethanol, 1.0 g of poly(methyl vinyl ether-alt-maleic acid) (PMVE-MA) (weight-average molecular weight (Mw)=about 1,980,000 from Sigma-Aldrich) was dispersed, and then a 1.7% aqueous sodium hydrogen carbonate solution was charged thereto, and the mixture was stirred for 30 minutes to prepare a second precursor layer forming liquid having a solid content concentration of a partial sodium salt of poly(methyl vinyl ether-alt-maleic acid) of 1.6% by mass and an ethanol/water ratio of 45/55. At this time, the amount of sodium hydrogen carbonate charged was adjusted so as to be 1.2 mol equivalent (that is, since two carboxyl groups exist in the repeating unit, the amount of sodium hydrogen carbonate to each carboxyl group is equivalent to 0.6 mol equivalent) with respect to the repeating unit of methyl vinyl ether-alt-maleic acid. Therefore, in the obtained partial sodium salt of poly(methyl vinyl ether-alt-maleic acid), about 60% of carboxyl groups (—COOH) in the poly(methyl vinyl ether-alt-maleic acid) is converted (neutralized) to a sodium salt (—COONa) (alkali (earth) metal salt percentage (%)=60%), and hereinafter, the partial sodium salt is referred to as “compound 1”.

The first precursor layer forming liquid prepared above was applied to a 1 mm thick press sheet (urethane sheet) of a thermoplastic polyurethane elastomer (PANDEX (registered trademark) T-2180, manufactured by DAC Covestro Polymer Co., Ltd.) with a wire bar (yarn count #12). The solvent was naturally dried to form a first precursor layer on the urethane sheet. Next, the second precursor layer forming liquid prepared above was applied onto the first precursor layer with a wire bar (yarn count #12). The solvent was naturally dried to form a second precursor layer on the first precursor layer. Furthermore, the second precursor layer (surface to which the second precursor layer forming liquid is applied) was irradiated with an electron beam using an electron beam irradiation device (EYE Compact EB (registered trademark) manufactured by IWASAKI ELECTRIC CO., LTD.) under the conditions of an acceleration voltage of 60 kV and an irradiation dose of 200 kGy in a nitrogen gas atmosphere to obtain sample 1. In the obtained sample 1, a first layer 102a (dry film thickness: about 2 μm) including p(DMAA-GMA) (3/1) and a second layer (dry film thickness: about 0.6 μm) including the compound 1 were sequentially formed on the urethane sheet.

### Example 2

Sample 2 was obtained in the same manner as in Example 1, except that p(DMAA-GMA) (6/1) (block copolymer 4) was used instead of p(DMAA-GMA) (3/1) in Example 1.

### Example 3

A sample 3 was obtained in the same manner as in Example 1, except that p(DMAA-GMA) (12/1) (block copolymer 1) was used instead of p(DMAA-GMA) (3/1) in Example 1.

### Example 4

Sample 4 was obtained in the same manner as in Example 1, except that p(DMAA-GMA) (35/1) (block copolymer 5) was used instead of p(DMAA-GMA) (3/1) in Example 1.

### Example 5

Sample 5 was obtained in the same manner as in Example 1, except that p(DMAA-GMA) (70/1) (block copolymer 6) was used instead of p(DMAA-GMA) (3/1) in Example 1.

### Example 6

Sample 6 was obtained in the same manner as in Example 1, except that p(PEGMA-GMA) (12/1) (block copolymer 8) was used instead of p(DMAA-GMA) (3/1) in Example 1.

### Example 7

In a mixed solution of 85 g of ethanol and 0.7 g of distilled water, 12 g of a methyl vinyl ether maleic anhydride copolymer (trade name: GANTREZ AN-169, (C₄H₂O₃·C₃H₆O)ₙ (n=430), manufactured by Ashland Japan Co., Ltd.) was charged and refluxed at 78° C. for 24 hours. The reaction product was reprecipitated by dropping the mixture into hexane after cooling the reflux liquid. Next, this precipitate was filtered off and then dried at 70° C. under reduced pressure for 3 days to obtain a reaction product of a methyl vinyl ether maleic anhydride copolymer (yield: 9 g). The degree of esterification (ratio of an ester moiety in a structural unit derived from maleic acid) of the reaction product of the methyl vinyl ether maleic anhydride copolymer thus obtained was measured by NMR and found to be 50%. The NMR measurement conditions are as follows. Hereinafter, the reaction product of the methyl vinyl ether maleic anhydride copolymer is also referred to as “half ethyl ester of methyl vinyl ether maleic anhydride copolymer”.
NMR instrument: Unity Plus NMR Spectrometer (manufactured by Varian)
Resonance frequency: 399.897 MHz
Integration: 8 times
Measurement solvent: Acetone-d⁶
Reference peak: 2.04 ppm (residual proton in Acetone-d⁶)
Sample concentration: 10 mg/0.75 mL Acetone-d⁶.

After 1.0 g of the half ethyl ester of the methyl vinyl ether maleic anhydride copolymer obtained above was dissolved in 24.0 g of acetone, a 1.7% aqueous sodium hydrogen carbonate solution was charged thereto, and the mixture was stirred for 30 minutes (alkali treatment) to prepare a second precursor layer forming liquid having a solid content concentration of the sodium salt of the methyl vinyl ether maleic anhydride copolymer half ethyl ester (“compound 2”) of 2% by mass, and an acetone/water ratio of 1/1. At this time, the amount of sodium hydrogen carbonate charged was adjusted so as to be 1.0 mol equivalent with respect to the repeating unit of the half ethyl ester of the methyl vinyl ether maleic anhydride copolymer. Therefore, all the carboxyl groups (—COOH) in the methyl vinyl ether maleic anhydride copolymer half ethyl ester were converted (neutralized) to (about 100%) sodium salt (—COONa) by the alkali treatment. Compound 2 is composed of structural units having the following structures.

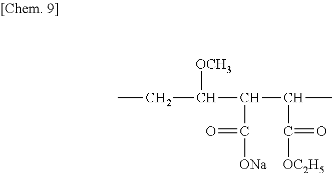

Sample 7 was obtained in the same manner as in Example 3 except that the second precursor layer forming liquid prepared above was used instead in Example 3.

### Example 8

A second precursor layer forming liquid was prepared in the same manner as in Example 1 except that the amount of sodium hydrogen carbonate charged was adjusted to 0.2 mol equivalent per the repeating unit of methyl vinyl ether-alt-maleic acid (corresponding to 0.1 mol equivalent with respect to the carboxyl group) in Example 1. Therefore, in the obtained partial sodium salt of poly(methyl vinyl ether-alt-maleic acid), about 10% of carboxyl groups (—COOH) in the poly(methyl vinyl ether-alt-maleic acid) is converted (neutralized) to a sodium salt (—COONa) (alkali (earth) metal salt percentage (%)=10%), and hereinafter, the partial sodium salt is also referred to as “compound 3”.

In the thorium salt, about 10% of carboxyl groups (—COOH) in poly(methyl vinyl ether-alt-maleic acid) are converted (neutralized) to a sodium salt (—COONa) (alkali (earth) metal salt percentage (%)=10%), and hereinafter, the thorium salt is also referred to as “compound 3”.

Sample 8 was obtained in the same manner as in Example 3 except that the second precursor layer forming liquid prepared above was used instead in Example 3.

### Example 9

A second precursor layer forming liquid was prepared in the same manner as in Example 1 except that the amount of sodium hydrogen carbonate charged was adjusted to 0.5 mol equivalent per the repeating unit of methyl vinyl ether-alt-maleic acid (corresponding to 0.25 mol equivalent with respect to the carboxyl group) in Example 1. In the obtained partial sodium salt of poly(methyl vinyl ether-alt-maleic acid), about 25% of carboxyl groups (—COOH) in the poly(methyl vinyl ether-alt-maleic acid) are converted (neutralized) to a sodium salt (—COONa) (alkali (earth) metal salt percentage (%)=25%), and hereinafter, the partial sodium salt is also referred to as “compound 4”.

Sample 9 was obtained in the same manner as in Example 3 except that the second precursor layer forming liquid prepared above was used instead in Example 3.

### Example 10

A second precursor layer forming liquid was prepared in the same manner as in Example 1 except that the amount of sodium hydrogen carbonate charged was adjusted to 2 mol equivalent per the repeating unit of methyl vinyl ether-alt-maleic acid (corresponding to 1 mol equivalent with respect to each carboxyl group) in Example 1. In the obtained partial sodium salt of poly(methyl vinyl ether-alt-maleic acid), all carboxyl groups (—COOH) (about 100%) in the poly(methyl vinyl ether-alt-maleic acid) are converted (neutralized) to a sodium salt (—COONa) (alkali (earth) metal salt percentage (%)=100%), and hereinafter, the partial sodium salt is also referred to as “compound 5”.

Sample 10 was obtained in the same manner as in Example 3 except that the second precursor layer forming liquid prepared above was used instead in Example 3.

### Example 11

Sample 11 was obtained in the same manner as in Example 1 except that a 1 mm thick press sheet of nylon (registered trademark) (nylon sheet) elastomer (ELG5660, manufactured by EMS) was used instead of the urethane sheet in Example 1.

### Example 12

Sample 12 was obtained in the same manner as in Example 2 except that a 1 mm thick press sheet of nylon (registered trademark) (nylon sheet) elastomer (ELG5660, manufactured by EMS) was used instead of the urethane sheet in Example 2.

### Example 13

Sample 13 was obtained in the same manner as in Example 3 except that a 1 mm thick press sheet of nylon (registered trademark) (nylon sheet) elastomer (ELG5660, manufactured by EMS) was used instead of the urethane sheet in Example 3.

### Example 14

Sample 14 was obtained in the same manner as in Example 7 except that a 1 mm thick press sheet of nylon (registered trademark) (nylon sheet) elastomer (ELG5660, manufactured by EMS) was used instead of the urethane sheet in Example 7.

### Example 15

Sample 15 was obtained in the same manner as in Example 5 except that a 1 mm thick press sheet of nylon (registered trademark) (nylon sheet) elastomer (ELG5660, manufactured by EMS) was used instead of the urethane sheet in Example 5.

### Example 16

Sample 16 was obtained in the same manner as in Example 6 except that a 1 mm thick press sheet of nylon (registered trademark) (nylon sheet) elastomer (ELG5660, manufactured by EMS) was used instead of the urethane sheet in Example 6.

### Example 17

Sample 17 was obtained in the same manner as in Example 3 except that the irradiation dose of the electron beam was changed to 30 kGy in Example 3.

### Example 18

Sample 18 was obtained in the same manner as in Example 3 except that the irradiation dose of the electron beam was changed to 100 kGy in Example 3.

### Example 19

Sample 19 was obtained in the same manner as in Example 3 except that the irradiation dose of the electron beam was changed to 350 kGy in Example 3.

### Example 20

Sample 20 was obtained in the same manner as in Example 3 except that the irradiation dose of the electron beam was changed to 500 kGy in Example 3.

### Comparative Example 1

A second precursor layer forming liquid was prepared in the same manner as in Example 1.

The second precursor layer forming liquid prepared above was applied to a 1 mm thick press sheet (urethane sheet) of a thermoplastic polyurethane elastomer (PANDEX (registered trademark) T-2180, manufactured by DAC Covestro Polymer Co., Ltd.) with a wire bar (yarn count #12). The solvent was naturally dried to form a second precursor layer on the urethane sheet. Next, the second precursor layer (surface to which the second precursor layer forming liquid is applied) was irradiated with an electron beam using an electron beam irradiation device (EYE Compact EB (registered trademark) manufactured by IWASAKI ELECTRIC CO., LTD.) under the conditions of an acceleration voltage of 60 kV and an irradiation dose of 200 kGy in a nitrogen gas atmosphere to obtain sample 21. In the obtained sample 21, a layer including the compound 1 (dry film thickness: about 0.6 μm) was formed on the urethane sheet.

### Comparative Example 2

A second precursor layer forming liquid was prepared in the same manner as in Example 7.

The second precursor layer forming liquid prepared above was applied to a 1 mm thick press sheet (urethane sheet) of a thermoplastic polyurethane elastomer (PANDEX (registered trademark) T-2180, manufactured by DAC Covestro Polymer Co., Ltd.) with a wire bar (yarn count #12). The solvent was naturally dried to form a second precursor layer on the urethane sheet. Next, the second precursor layer (surface to which the second precursor layer forming liquid is applied) was irradiated with an electron beam using an electron beam irradiation device (EYE Compact EB (registered trademark) manufactured by IWASAKI ELECTRIC CO., LTD.) under the conditions of an acceleration voltage of 60 kV and an irradiation dose of 200 kGy in a nitrogen gas atmosphere to obtain sample 22. In the obtained sample 22, a layer including the compound 2 (dry film thickness: about 0.7 μm) was formed on the urethane sheet.

### Comparative Example 3

Sample 23 was obtained in the same manner as in Comparative Example 1 except that a 1 mm thick press sheet of nylon (registered trademark) (nylon sheet) elastomer (ELG5660, manufactured by EMS) was used instead of the urethane sheet in Comparative Example 1.

### Comparative Example 4

Sample 24 was obtained in the same manner as in Comparative Example 2 except that a 1 mm thick press sheet of nylon (registered trademark) (nylon sheet) elastomer (ELG5660, manufactured by EMS) was used instead of the urethane sheet in Comparative Example 2.

### Comparative Example 5

Sample 25 was obtained in the same manner as in Example 1, except that p(DMAA-GMA) (2/1) (block copolymer 2) was used instead of p(DMAA-GMA) (3/1) in Example 1.

### Comparative Example 6

Sample 26 was obtained in the same manner as in Example 1, except that p(DMAA-GMA) (100/1) (block copolymer 7) was used instead of p(DMAA-GMA) (3/1) in Example 1.

### Comparative Example 7

Sample 27 was obtained in the same manner as in Example 1, except that poly(N,N-dimethylacrylamide) (manufactured by Scientific Polymer, weight-average molecular weight (Mw)=100,000) (pDMAA) was used instead of p(DMAA-GMA) (3/1) in Example 1.

### Comparative Example 8

Sample 28 was obtained in the same manner as in Example 7, except that poly(N,N-dimethylacrylamide) (manufactured by Scientific Polymer, weight-average molecular weight (Mw)=100,000) (pDMAA) was used instead of p(DMAA-GMA) (12/1) in Example 7.

### Comparative Example 9

Sample 29 was obtained in the same manner as in Example 7, except that p (MEA-GMA) (12/1) (block copolymer 9) was used instead of p(DMAA-GMA) (12/1) in Example 7.

### Comparative Example 10

Polyvinylpyrrolidone (manufactured by Tokyo Chemical Industry Co., Ltd., K90, weight-average molecular weight=360,000) (PVP) was dissolved in ethanol so as to be 6% by mass to prepare a first precursor layer forming liquid.

Sample 30 was obtained in the same manner as in Example 7 except that the first precursor layer forming liquid prepared above was used instead in Example 7. In this example, since polyvinylpyrrolidone is insoluble in acetone, ethanol was used instead of acetone to measure the swelling ratio.

### Comparative Example 11

Sample 31 was obtained in the same manner as in Example 11, except that p (MEA-GMA) (12/1) (block copolymer 9) was used in place of p(DMAA-GMA) (3/1) in Example 11.

### Comparative Example 12

Sample 32 was obtained in the same manner as in Example 14, except that poly(N,N-dimethylacrylamide) (manufactured by Scientific Polymer, weight-average molecular weight (Mw)=100,000) (pDMAA) was used instead of p(DMAA-GMA) (12/1) in Example 14.

### Comparative Example 13

A first precursor layer forming liquid was prepared in the same manner as in Comparative Example 10.

Sample 33 was obtained in the same manner as in Example 14 except that the first precursor layer forming liquid prepared above was used instead in Example 14.

### Comparative Example 14

Sample 34 was obtained in the same manner as in Example 11, except that polyethylene glycol (manufactured by Wako Pure Chemical Industries, Ltd., average molecular weight=3,600,000 to 4,000,000) (PEG) was used instead of p(DMAA-GMA) (3/1) in Example 11.

### Comparative Example 15

Sample 35 was obtained in the same manner as in Comparative Example 12 except that the irradiation dose of the electron beam was changed to 500 kGy in Comparative Example 12.

### Comparative Example 16

Sample 36 was obtained in the same manner as in Comparative Example 13 except that the irradiation dose of the electron beam was changed to 500 kGy in Comparative Example 13.

### Comparative Example 17

A second precursor layer forming liquid was prepared in the same manner as in Example 1.

Sample 37 was obtained in the same manner as in Comparative Example 16 except that the second precursor layer forming liquid prepared above was used instead in Comparative Example 16.

### Comparative Example 18

Sodium hyaluronate (manufactured by Tokyo Chemical Industry Co., Ltd., derived from cock's comb) was dissolved in water so as to be 0.25% by mass, thereby preparing a second precursor layer forming liquid.

Sample 38 was obtained in the same manner as in Example 3, except that the second precursor layer forming liquid prepared above was used instead and a wire bar (yarn count #36) was used instead in Example 3. In the obtained sample 38, a first layer 102a (dry film thickness: about 2.0 μm) including p(DMAA-GMA) (12/1) and a second layer (dry film thickness: about 0.3 μm) including sodium hyaluronate were sequentially formed on the urethane sheet.

### Comparative Example 19

A second precursor layer forming liquid was prepared by dissolving polyacrylamide (weight average molecular weight (Mw)=400,000-800,000, manufactured by Tokyo Chemical Industry Co., Ltd.) in water so as to be 1% by mass.

Sample 39 was obtained in the same manner as in Example 3, except that the second precursor layer forming liquid prepared above was used instead and a wire bar (yarn count #20) was used instead in Example 3. In the obtained sample 39, a first layer 102a (dry film thickness: about 2.0 μm) including p(DMAA-GMA) (12/1) and a second layer (dry film thickness: about 0.6 μm) including polyacrylamide were sequentially formed on the urethane sheet.

### Comparative Example 20

Sodium polyacrylate (manufactured by Wako Pure Chemical Industries, Ltd., polymerization degree 30,000 to 40,000) was dissolved in water so as to be 0.25% by mass to prepare a second precursor layer forming liquid.

Sample 40 was obtained in the same manner as in Example 3, except that the second precursor layer forming liquid prepared above was used instead and a wire bar (yarn count #36) was used instead in Example 3. In the obtained sample 40, a first layer 102a (dry film thickness: about 2.0 μm) including p(DMAA-GMA) (12/1) and a second layer (dry film thickness: about 0.3 μm) including sodium polyacrylate were sequentially formed on the urethane sheet.

### Comparative Example 21

Sample 41 was obtained in the same manner as in Comparative Example 18 except that the irradiation dose of the electron beam was changed to 500 kGy in Comparative Example 18.

### Comparative Example 22

Sample 42 was obtained in the same manner as in Comparative Example 19 except that the irradiation dose of the electron beam was changed to 500 kGy in Comparative Example 19.

### Comparative Example 23

Sample 43 was obtained in the same manner as in Comparative Example 20 except that the irradiation dose of the electron beam was changed to 500 kGy in Comparative Example 20.

The material of the base material of samples 1 to 43 obtained above, the first copolymer of the first layer 102a (first precursor layer), the second copolymer of the second layer (second precursor layer), and the electron beam irradiation conditions are summarized in the following Table 1. In Table 1 below, the “irradiation condition (kV-kGy)” indicates the acceleration voltage (kV) and the irradiation dose (kGy). For example, the irradiation condition of the electron beam in Example 1 indicates that the acceleration voltage is 60 kV and the irradiation dose is 200 kGy.

In addition, the lubricating property and lubrication retaining property (durability) of the samples 1 to 43 obtained above were evaluated according to the following method. The results are shown in Table 1 below.

### [Sensory Evaluation]

Sensory evaluation was performed on the samples 1 to 20 and 23 to 43 obtained above according to the following method. Sample 21 (Comparative Example 1) and sample 22 (Comparative Example 2) could not be evaluated because repellency was observed during applying.

The entire medical device was immersed in water with the second layer (surface to which the second precursor layer forming liquid is applied) of each sample facing upward. After 1 minute, the second layer was rubbed back and forth 30 times with the pad of a finger in a state of being immersed in water, and slipperiness after 1 reciprocation (“slipping” in the following Table 1) and slipperiness after 30 reciprocations (“durability” in the following Table 1) were evaluated. The rubbing strength and speed were as similar as possible between the samples.

The slipperiness and durability after 1 reciprocation (slipperiness after 30 reciprocations) were evaluated according to the following criteria.

### (Evaluation Criteria for Slipperiness)

⊙: Slides very well
o: Slides well
Δ: Sliding but sticking
x: Almost no slip
xx: No slippage at all

### (Evaluation Criteria of Durability)

⊙: No change in slipperiness was observed.
o: No change in slipperiness was observed.
(The slipperiness at 30 reciprocations is slightly deteriorated as compared with 1 reciprocation)
Δ: Slipperiness deteriorates from 20 reciprocations
x: Slipperiness deteriorates with 3 to 10 reciprocations.
xx: Almost no slippage occurs with 1 to 2 reciprocations.

### [Evaluation of Sliding Durability]

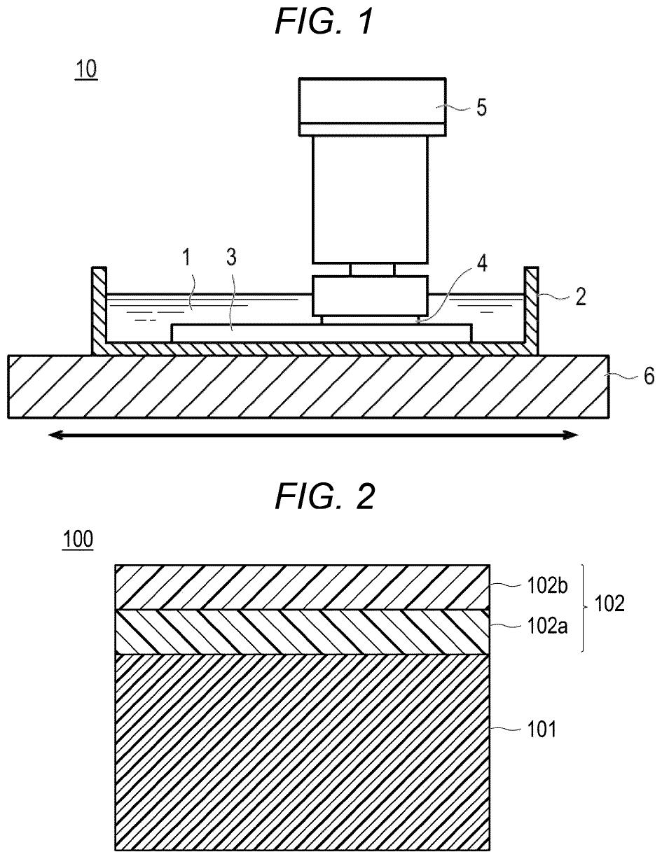

For the medical device in which both the slipperiness and the durability were evaluated as ⊙ or o in the [Sensory evaluation], the sliding property (slipperiness) and the sliding durability (lubrication retaining property) of the lubricating layer of each sample were further evaluated according to the following method using a friction tester (Handy Livo Master TL201 manufactured by Trinity-Lab Inc.) 10 shown in FIG. 1.

That is, sample 3 was fixed in the petri dish 2 with the second layer (surface to which the second precursor layer forming liquid is applied) of each sample 3 facing up and immersed in water 1 at a height in which the entire sample 3 was immersed. The petri dish 2 was placed on the moving table 6 of the friction tester 10 illustrated in FIG. 1. A terminal (φ 10 mm) 4 made of a hydrogenated styrene thermoplastic elastomer (SEBS) was brought into contact with sample 3, and a load 5 of 200 g was applied onto the terminal 4. The moving table 6 was horizontally reciprocated 50 times at a sliding distance of 15 mm and a sliding speed of 16.7 mm/sec and sliding resistance values (gf) at the 1st reciprocation, the 5th reciprocation, the 10th reciprocation, the 20th reciprocation, the 30th reciprocation, and the 50th reciprocation were measured.

TABLE 1

| | | | | Irradiation | | | | | | | | | |
|---|---|---|---|---|---|---|---|---|---|---|---|---|---|
| | | | | condition | | Sensory | | | | | | | |
| | Base | First | Second | (kV- | Swelling | evaluation | | Sliding resistance value (gf) | | | | | |
| | material | layer | layer | kGy) | ratio | Slipping | Durability | 1 | 5 | 10 | 20 | 30 | 50 |
| Example 1 | Urethane | p (DMAA- | Compound 1 | 60- | 220% | ⊙ | ⊙ | 7.21 | 7.33 | 7.44 | 8.55 | 8.63 | 9.07 |
| | | GMA) | | 200 | | | | | | | | | |
| | | (3/1) | | | | | | | | | | | |
| Example 2 | Urethane | p (DMAA- | Compound 1 | 60- | 320% | ⊙ | ⊙ | 3.61 | 6.22 | 7.59 | 8.11 | 8.31 | 8.55 |
| | | GMA) | | 200 | | | | | | | | | |
| | | (6/1) | | | | | | | | | | | |
| Example 3 | Urethane | p (DMAA- | Compound 1 | 60- | 670% | ⊙ | ⊙ | 2.73 | 2.89 | 3.01 | 3.26 | 3.52 | 3.69 |
| | | GMA) | | 200 | | | | | | | | | |
| | | (12/1) | | | | | | | | | | | |
| Example 4 | Urethane | p (DMAA- | Compound 1 | 60- | 780% | ⊙ | ⊙ | 2.79 | 2.91 | 3.04 | 3.11 | 3.19 | 3.21 |
| | | GMA) | | 200 | | | | | | | | | |
| | | (35/1) | | | | | | | | | | | |
| Example 5 | Urethane | p (DMAA- | Compound 1 | 60- | 900% | ⊙ | ⊙ | 2.25 | 2.26 | 2.19 | 2.21 | 2.25 | 2.29 |
| | | GMA) | | 200 | | | | | | | | | |
| | | (70/1) | | | | | | | | | | | |
| Example 6 | Urethane | p (PEGMA- | Compound 1 | 60- | 190% | ⊙ | ⊙ | 8.02 | 8.63 | 8.69 | 8.79 | 8.80 | 8.88 |
| | | GMA) | | 200 | | | | | | | | | |
| | | (12/1) | | | | | | | | | | | |
| Example 7 | Urethane | p (DMAA- | Compound 2 | 60- | 670% | ⊙ | ⊙ | 3.08 | 2.96 | 3.04 | 3.33 | 3.60 | 3.99 |
| | | GMA) | | 200 | | | | | | | | | |
| | | (12/1) | | | | | | | | | | | |
| Example 8 | Urethane | p (DMAA- | Compound 3 | 60- | 670% | ⊙ | ⊙ | 6.08 | 6.05 | 6.08 | 6.27 | 6.29 | 6.48 |
| | | GMA) | | 200 | | | | | | | | | |
| | | (12/1) | | | | | | | | | | | |
| Example 9 | Urethane | p (DMAA- | Compound 4 | 60- | 670% | ⊙ | ⊙ | 4.85 | 4.75 | 4.79 | 4.93 | 4.90 | 4.88 |
| | | GMA) | | 200 | | | | | | | | | |
| | | (12/1) | | | | | | | | | | | |
| Example 10 | Urethane | p (DMAA- | Compound 5 | 60- | 670% | ⊙ | ⊙ | 3.25 | 3.61 | 3.79 | 3.78 | 3.74 | 3.77 |
| | | GMA) | | 200 | | | | | | | | | |
| | | (12/1) | | | | | | | | | | | |
| Example 11 | Nylon | p (DMAA- | Compound 1 | 60- | 220% | ⊙ | ⊙ | 6.11 | 6.34 | 6.50 | 7.13 | 7.55 | 8.58 |
| | | GMA) | | 200 | | | | | | | | | |
| | | (3/1) | | | | | | | | | | | |
| Example 12 | Nylon | p (DMAA- | Compound 1 | 60- | 320% | ⊙ | ⊙ | 3.27 | 3.36 | 3.46 | 3.58 | 3.68 | 3.97 |
| | | GMA) | | 200 | | | | | | | | | |
| | | (6/1) | | | | | | | | | | | |
| Example 13 | Nylon | p (DMAA- | Compound 1 | 60- | 670% | ⊙ | ⊙ | 2.05 | 2.00 | 1.99 | 1.91 | 1.88 | 1.89 |
| | | GMA) | | 200 | | | | | | | | | |
| | | (12/1) | | | | | | | | | | | |
| Example 14 | Nylon | p (DMAA- | Compound 2 | 60- | 670% | ⊙ | ⊙ | 2.19 | 2.09 | 1.97 | 2.02 | 2.06 | 2.12 |
| | | GMA) | | 200 | | | | | | | | | |
| | | (12/1) | | | | | | | | | | | |
| Example 15 | Nylon | p (DMAA- | Compound 1 | 60- | 900% | ⊙ | ⊙ | 2.55 | 2.59 | 2.48 | 2.53 | 2.69 | 2.75 |
| | | GMA) | | 200 | | | | | | | | | |
| | | (70/1) | | | | | | | | | | | |
| Example 16 | Nylon | p (PEGMA- | Compound 1 | 60- | 190% | ⊙ | ⊙ | 8.09 | 8.38 | 8.52 | 8.81 | 8.92 | 9.11 |
| | | GMA) | | 200 | | | | | | | | | |
| | | (12/1) | | | | | | | | | | | |
| Example 17 | Urethane | p (DMAA- | Compound 1 | 60- | 670% | ⊙ | ⊙ | 1.49 | 1.51 | 1.77 | 3.20 | 3.65 | 4.82 |
| | | GMA) | | 30 | | | | | | | | | |
| | | (12/1) | | | | | | | | | | | |
| Example 18 | Urethane | p (DMAA- | Compound 1 | 60- | 670% | ⊙ | ⊙ | 2.35 | 2.44 | 2.69 | 2.72 | 2.77 | 2.83 |
| | | GMA) | | 100 | | | | | | | | | |
| | | (12/1) | | | | | | | | | | | |
| Example 19 | Urethane | p (DMAA- | Compound 1 | 60- | 670% | ⊙ | ⊙ | 4.76 | 4.84 | 4.82 | 4.89 | 4.81 | 4.82 |
| | | GMA) | | 350 | | | | | | | | | |
| | | (12/1) | | | | | | | | | | | |
| Example 20 | Urethane | p (DMAA- | Compound 1 | 60- | 670% | ⊙ | ⊙ | 8.22 | 8.25 | 8.11 | 8.15 | 8.07 | 8.05 |
| | | GMA) | | 500 | | | | | | | | | |
| | | (12/1) | | | | | | | | | | | |
| Comparative | Urethane | — | Compound 1 | 60- | — | — | — | | | | | | |
| Example 1 | | | | 200 | | | | | | | | | |
| Comparative | Urethane | — | Compound 2 | 60- | — | — | — | | | | | | |
| Example 2 | | | | 200 | | | | | | | | | |
| Comparative | Nylon | — | Compound 1 | 60- | — | ◯ | X | | | | | | |
| Example 3 | | | | 200 | | | | | | | | | |
| Comparative | Nylon | — | Compound 2 | 60- | — | ◯ | X | | | | | | |
| Example 4 | | | | 200 | | | | | | | | | |
| Comparative | Urethane | p (DMAA- | Compound 1 | 60- | 70% | ⊙ | ◯ | 1.68 | 3.49 | 5.34 | 7.96 | 9.61 | — |
| Example 5 | | GMA) | | 200 | | | | | | | | | |
| | | (2/1) | | | | | | | | | | | |
| Comparative | Urethane | p (DMAA- | Compound 1 | 60- | Dissolved | ⊙ | ◯ | 2.61 | 2.47 | 2.48 | 3.01 | 4.14 | 17.51 |
| Example 6 | | GMA) | | 200 | | | | | | | | | |
| | | (100/1) | | | | | | | | | | | |
| Comparative | Urethane | pDMAA | Compound 1 | 60- | Dissolved | ⊙ | X | | | | | | |
| Example 7 | | | | 200 | | | | | | | | | |
| Comparative | Urethane | pDMAA | Compound 2 | 60- | Dissolved | ⊙ | X | | | | | | |
| Example 8 | | | | 200 | | | | | | | | | |
| Comparative | Urethane | p (MEA- | Compound 2 | 60- | 1.2% | ⊙ | X | | | | | | |
| Example 9 | | GMA) | | 200 | | | | | | | | | |
| | | (12/1) | | | | | | | | | | | |
| Comparative | Urethane | PVP | Compound 2 | 60- | Dissolved | ⊙ | X | | | | | | |
| Example 10 | | | | 200 | | | | | | | | | |
| Comparative | Nylon | p (MEA- | Compound 1 | 60- | 1.2% | ⊙ | Δ | | | | | | |
| Example 11 | | GMA) | | 200 | | | | | | | | | |
| | | (12/1) | | | | | | | | | | | |
| Comparative | Nylon | pDMAA | Compound 2 | 60- | Dissolved | ⊙ | X | | | | | | |
| Example 12 | | | | 200 | | | | | | | | | |
| Comparative | Nylon | PVP | Compound 2 | 60- | Dissolved | ⊙ | X | | | | | | |
| Example 13 | | | | 200 | | | | | | | | | |
| Comparative | Nylon | PEG | Compound 1 | 60- | Dissolved | ⊙ | X | | | | | | |
| Example 14 | | | | 200 | | | | | | | | | |
| Comparative | Nylon | pDMAA | Compound 2 | 60- | Dissolved | ⊙ | Δ | | | | | | |
| Example 15 | | | | 500 | | | | | | | | | |
| Comparative | Nylon | PVP | Compound 2 | 60- | Dissolved | ⊙ | Δ | | | | | | |
| Example 16 | | | | 500 | | | | | | | | | |
| Comparative | Nylon | PVP | Compound 1 | 60- | Dissolved | ⊙ | Δ | | | | | | |
| Example 17 | | | | 500 | | | | | | | | | |
| Comparative | Urethane | p (DMAA- | Sodium | 60- | 670% | ◯ | Δ | | | | | | |
| Example 18 | | GMA) | hyaluronate | 200 | | | | | | | | | |
| | | (12/1) | | | | | | | | | | | |
| Comparative | Urethane | p (DMAA- | Polyacryl | 60- | 670% | ◯ | X | | | | | | |
| Example 19 | | GMA) | amide | 200 | | | | | | | | | |
| | | (12/1) | | | | | | | | | | | |
| Comparative | Urethane | p (DMAA- | Sodium | 60- | 670% | ◯ | X | | | | | | |
| Example 20 | | GMA) | polyacrylate | 200 | | | | | | | | | |
| | | (12/1) | | | | | | | | | | | |
| Comparative | Urethane | p (DMAA- | Sodium | 60- | 670% | ◯ | Δ | | | | | | |
| Example 21 | | GMA) | hyaluronate | 500 | | | | | | | | | |
| | | (12/1) | | | | | | | | | | | |
| Comparative | Urethane | p (DMAA- | Polyacryl | 60- | 670% | ◯ | X | | | | | | |
| Example 22 | | GMA) | amide | 500 | | | | | | | | | |
| | | (12/1) | | | | | | | | | | | |
| Comparative | Urethane | P(DMAA- | Sodium | 60- | 670% | ◯ | X | | | | | | |
| Example 23 | | GMA)(12/1) | hyaluronate | 500 | | | | | | | | | |

From the results in Table 1, it is found that the samples 1 to 20 of Example are superior to the samples 21 to 43 of Comparative Example in both slipperiness (slipping and sliding resistance value at the 1st reciprocation in Table 1) and durability (durability and sliding resistance value at the 50th reciprocation in Table 1).

Sample 25 of Comparative Example 5 and sample 26 of Comparative Example 6 were excellent in slipperiness and durability in the sensory evaluation but was significantly inferior in durability in the severer [see [Evaluation of sliding durability]].

The detailed description above describes embodiments of a medical device representing examples of the new medical device disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

### REFERENCE SIGNS LIST

1 Water
2 Petri dish
3 Sample (medical device)
4 Terminal
5 Load
6 Moving table
10 Friction tester
101 Base layer
101a Base layer core portion
101b Base material surface layer
102 Lubricating layer
102a First layer
102b Second layer
100 Medical device

What is claimed is:

1. A medical device comprising:
a base layer;
two additional layers that together constitute a lubricating layer and that are successively applied to the base layer so that the two additional layers overlie the base layer, the two additional layers that are successively applied to the base layer comprising a first layer and a second layer, at least a part of the first layer being in contact with the base layer and being between the base layer and the second layer, and at least a part of the second layer being in contact with the first layer;
the first layer comprising a first copolymer, the first copolymer comprising a structural unit derived from a hydrophilic monomer and a structural unit having an epoxy group, the first layer having a swelling ratio of at least 190% and less than 1000%; and
the second layer comprising a second copolymer, the second copolymer comprising a structural unit having an alkyl vinyl ether group and a structural unit having a carboxyl group or a salt or ester thereof.

2. The medical device according to claim 1, wherein the base layer comprises a metal material, a polymer material or a ceramic material.

3. The medical device according to claim 1, wherein the first copolymer comprises the structural unit derived from the hydrophilic monomer and the structural unit having the epoxy group in a molar ratio of 3:1 to 70:1.

4. The medical device according to claim 1, wherein the hydrophilic monomer comprises an amino group (—N(R1)(R2), wherein R1 and R2 each independently denote a hydrogen atom or an unsubstituted or substituted alkyl group having 1 to 4 carbon atoms), or an alkylene glycol chain.

5. A medical device comprising:
a base layer;
a lubricating layer having a first layer formed on at least a part of the base layer and a second layer formed on at least part of the first layer;
the first layer comprising a first copolymer, the first copolymer comprising a structural unit derived from a hydrophilic monomer and a structural unit having an epoxy group, the first layer having a swelling ratio of more than 70% and less than 1000%; and
the second layer comprising a second copolymer, the second copolymer comprising a structural unit having an alkyl vinyl ether group and a structural unit having a carboxyl group or a salt or ester thereof.

6. The medical device according to claim 5, wherein the first copolymer comprises the structural unit derived from the hydrophilic monomer and the structural unit having the epoxy group in a molar ratio of 3:1 to 70:1.

7. The medical device according to claim 5, wherein the hydrophilic monomer comprises an amino group —N(R1)(R2), wherein R1 and R2 each independently denote a hydrogen atom or an unsubstituted or substituted alkyl group having 1 to 4 carbon atoms), or an alkylene glycol chain.

8. The medical device according to claim 5, wherein the hydrophilic monomer comprises at least one monomer selected from the group consisting of acrylamide, N-methyl acrylamide, N,N-dimethylacrylamide (DMAA), N-ethylacrylamide, N,N-diethylacrylamide (DEAA), N,N-dimethylaminopropylacrylamide, N,N-dimethylaminopropylmethacrylamide, 2-acrylamide-2-methylpropanesulfonic acid, N-(2-hydroxyethyl) acrylamide, N-(2-hydroxypropyl) acrylamide, N-(2-hydroxybutyl) acrylamide, polyethylene glycol monoacrylate, polyethylene glycol monomethacrylate, alkoxypolyethylene glycol monoacrylate, and alkoxypolyethylene glycol monomethacrylate.

9. The medical device according to claim 5, wherein the structural unit having the epoxy group is a structural unit derived from at least one monomer selected from the group consisting of glycidyl acrylate, glycidyl methacrylate (GMA), 3,4-epoxycyclohexyl methyl acrylate, 3,4-epoxycyclohexyl methyl methacrylate, β-methyl glycidyl acrylate, and β-methyl glycidyl methacrylate.

10. The medical device according to claim 5, wherein the structural unit having the carboxyl group or the salt or ester thereof comprises at least one of:
a structural unit represented by formula (1): 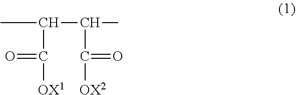
wherein X¹ denotes a hydrogen atom, an alkali metal, or an alkaline earth metal, and X² denotes a hydrogen atom, an alkali metal, an alkaline earth metal, a straight chain alkyl group having 1 to 24 carbon atoms, or a branched chain alkyl group having 3 to 24 carbon atoms; and
a structural unit represented by formula (2): 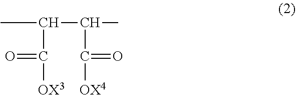
wherein X³ denotes a hydrogen atom, an alkali metal, or an alkaline earth metal, and X⁴ denotes a hydrogen atom, an alkali metal, or an alkaline earth metal.

11. The medical device according to claim 5, wherein the structural unit having the alkyl vinyl ether group is represented by formula (3): 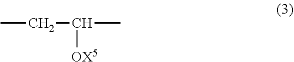
wherein X⁵ denotes a straight chain alkyl group having 1 to 24 carbon atoms or a branched chain alkyl group having 3 to 24 carbon atoms.

12. The medical device according to claim 5, wherein the lubricating layer comprises a region in which the first copolymer and the second copolymer are bonded to each other.

13. The medical device according to claim 5, wherein the medical device is a catheter, a stent, or a guide wire.

14. A method for manufacturing a medical device, the method comprising:
applying a first coating liquid comprising a first copolymer, the first polymer comprising a structural unit derived from a hydrophilic monomer and a structural unit having an epoxy group, and a solvent to at least a part of a base layer to form a first precursor layer on at least part of the base layer;
applying a second coating liquid comprising a second copolymer, the second copolymer comprising a structural unit having an alkyl vinyl ether group and a structural unit having a carboxyl group or a salt or ester thereof, and a solvent to at least part of the first precursor layer to obtain an intermediate laminated body, in which a second precursor layer is formed on at least part of the first precursor layer; and
irradiating the intermediate laminated body with an electron beam.

15. The method according to claim 14, wherein irradiating the intermediate laminated body with an electron beam comprises irradiating the intermediate laminated body at an irradiation dose of 30 to 500 kGy.

16. The method according to claim 14, wherein the first copolymer comprises the structural unit derived from the hydrophilic monomer and the structural unit having the epoxy group in a molar ratio of 3:1 to 70:1.

17. The method according to claim 14, wherein the hydrophilic monomer comprises an amino group (—N(R1)(R2), wherein R1 and R2 each independently denote a hydrogen atom or an unsubstituted or substituted alkyl group having 1 to 4 carbon atoms), or an alkylene glycol chain.

18. The method according to claim 14, wherein the hydrophilic monomer comprises at least one monomer selected from the group consisting of acrylamide, N-methyl acrylamide, N,N-dimethylacrylamide (DMAA), N-ethylacrylamide, N,N-diethylacrylamide (DEAA), N,N-dimethylaminopropylacrylamide, N,N-dimethylaminopropylmethacrylamide, 2-acrylamide-2-methylpropanesulfonic acid, N-(2-hydroxyethyl) acrylamide, N-(2-hydroxypropyl) acrylamide, N-(2-hydroxybutyl) acrylamide, polyethylene glycol monoacrylate, polyethylene glycol monomethacrylate, alkoxypolyethylene glycol monoacrylate, and alkoxypolyethylene glycol monomethacrylate.

19. The method according to claim 14, wherein the structural unit having the epoxy group is a structural unit derived from at least one monomer selected from the group consisting of glycidyl acrylate, glycidyl methacrylate (GMA), 3,4-epoxycyclohexyl methyl acrylate, 3,4-epoxycyclohexyl methyl methacrylate, β-methyl glycidyl acrylate, and β-methyl glycidyl methacrylate.

20. The method according to claim 14, wherein the structural unit having the carboxyl group or the salt or ester thereof comprises at least one of:
a structural unit represented by formula (1): 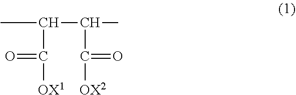
wherein X¹ denotes a hydrogen atom, an alkali metal, or an alkaline earth metal, and X² denotes a hydrogen atom, an alkali metal, an alkaline earth metal, a straight chain alkyl group having 1 to 24 carbon atoms, or a branched chain alkyl group having 3 to 24 carbon atoms; and
a structural unit represented by formula (2): 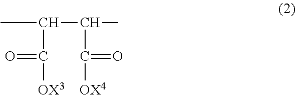
wherein X³ denotes a hydrogen atom, an alkali metal, or an alkaline earth metal, and X⁴ denotes a hydrogen atom, an alkali metal, or an alkaline earth metal.
